SEQUENCE LISTING (18 sequences)

SEQ ID NO: 1 - protein, 232 aa, homo sapiens
  misc_feature: Human VEGF-A precursor
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 2 - protein, 206 aa, homo sapiens
  misc_feature: Human VEGF-A, isoform 206
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 3 - protein, 189 aa, homo sapiens
  misc_feature: Human VEGF-A, isoform 189
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVPCGPCSERRKHLFVQDPQTCKCSCKNTDSRCKARQLELNERTCRCDKPRR

SEQ ID NO: 4 - protein, 165 aa, homo sapiens
  misc_feature: Human VEGF-A, isoform 165
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 5 - protein, 121 aa, homo sapiens
  misc_feature: Human VEGF-A, isoform 121
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENCDKPRR

SEQ ID NO: 6 - protein, 183 aa, homo sapiens
  misc_feature: Human VEGF-A, isoform 183
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRPCGPCSERRKHLFVQDPQTCKCSCKNTDSRCKARQLELNERTCRCDKPRR

SEQ ID NO: 7 - protein, 148 aa, homo sapiens
  misc_feature: Human VEGF-A, isoform 148
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKM

SEQ ID NO: 8 - protein, 145 aa, homo sapiens
  misc_feature: Human VEGF-A, isoform 145
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVCDKPRR

SEQ ID NO: 9 - protein, 207 aa, Homo sapiens
  misc_feature: Human VEGF-B, precursor
MSPLLRRLLLAALLQLAPAQAPVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDRAATPHHRPQPRSVPGWDSAPGAPSPADITHPTPAPGPSAHAAPSTTSALTPGPAAAAADAAASSVAKGGA

SEQ ID NO: 10 - protein, 419 aa, Homo sapiens
  misc_feature: Human VEGF-C, precursor
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS

SEQ ID NO: 11 - protein, 354 aa, Homo sapiens
  misc_feature: Human VEGF-D, precursor
MYREWVVVNVFMMLYVQLVQGSSNEHGPVKRSSQSTLERSEQQIRAASSLEELLRITHSEDWKLWRCRLRLKSFTSMDSRSASHRSTRFAATFYDIETLKVIDEEWQRTQCSPRETCVEVASELGKSTNTFFKPPCVNVFRCGGCCNEESLICMNTSTSYISKQLFEISVPLTSVPELVPVKVANHTGCKCLPTAPRHPYSIIRRSIQIPEEDRCSHSKKLCPIDMLWDSNKCKCVLQEENPLAGTEDHSHLQEPALCGPHMMFDEDRCECVCKTPCPKDLIQHPKNCSCFECKESLETCCQKHKLFHPDTCSCEDRCPFHTRPCASGKTACAKHCRFPKEKRAAQGPHSRKNP

SEQ ID NO: 12 - protein, 221 aa, Homo sapiens
  misc_feature: hPIGF, precursor
MPVMRLFPCFLQLLAGLALPAVPPQQWALSAGNGSSEVEVVPFQEVWGRSYCRALERLVDVVSEYPSEVEHMFSPSCVSLLRCTGCCGDENLHCVPVETANVTMQLLKIRSGDRPSYVELTFSQHVRCECRHSPGRQSPDMPGDFRADAPSFLPPRRSLPMLFRMEWGCALTGSQSAVWPSSPVPEEIPRMHPGRNGKKQQRKPLREKMKPERCGDAVPRR

SEQ ID NO: 13 - protein, 214 aa, mus musculus
  misc_feature: Murine VEGF, precursor
MNFLLSWVHWTLALLLYLHHAKWSQAAPTTEGEQKSHEVIKFMDVYQRSYCRPIETLVDIFQEYPDEIEYIFKPSCVPLMRCAGCCNDEALECVPTSESNITMQIMRIKPHQSQHIGEMSFLQHSRCECRPKKDRTKPEKKSVRGKGKGQKRKRKKSRFKSWSVHCEPCSERRKHLFVQDPQTCKCSCKNTDSRCKARQLELNERTCRCDKPRR

SEQ ID NO: 14 - protein, 190 aa, mus musculus
  misc_feature: Murine VEGF, isoform 1
MNFLLSWVHWTLALLLYLHHAKWSQAAPTTEGEQKSHEVIKFMDVYQRSYCRPIETLVDIFQEYPDEIEYIFKPSCVPLMRCAGCCNDEALECVPTSESNITMQIMRIKPHQSQHIGEMSFLQHSRCECRPKKDRTKPENHCEPCSERRKHLFVQDPQTCKCSCKNTDSRCKARQLELNERTCRCDKPRR

SEQ ID NO: 15 - protein, 146 aa, mus musculus
  misc_feature: Murine VEGF, isoform 2
MNFLLSWVHWTLALLLYLHHAKWSQAAPTTEGEQKSHEVIKFMDVYQRSYCRPIETLVDIFQEYPDEIEYIFKPSCVPLMRCAGCCNDEALECVPTSESNITMQIMRIKPHQSQHIGEMSFLQHSRCECRPKKDRTKPEKCDKPRR

SEQ ID NO: 16 - protein, 15 aa, Clostridium tetani
  misc_feature: Tetanus toxoid P2 epitope
QYIKANSKFIGITEL

SEQ ID NO: 17 - protein, 21 aa, Clostridium tetani
  misc_feature: Tetanus toxoid P30 epitope
FNNFTVSFWLRVPKVSASHLE

SEQ ID NO: 18 - protein, 13 aa, Artificial sequence
  Artificial pan DR binding amino acid sequence
AKFVAAWTLKAAA

(19) United States
(12) Patent Application Publication
(10) Pub. No.: US 2007/0184023 A1
(43) Pub. Date: Aug. 9, 2007

(54) Method for down-regulation of vegf

(75) Inventors: Peter Birk Rasmussen, Horsholm (DK); Florence Dal Degan, Horsholm (DK); Valery Renard, Horsholm (DK); Steen Klysner, Horsholm (DK); Birgitte Volck, Horsholm (DK)

Correspondence Address: BIRCH STEWART KOLASCH & BIRCH, PO BOX 747, FALLS CHURCH, VA, 22040-0747, US

(73) Assignee: Pharmexa A/S, Horsholm (DK)

(21) Appl. No.: 10/577,489

(22) PCT Filed: Oct. 28, 2004
(86) PCT No.: PCT/DK04/00741

§ 371 Date: Dec. 20, 2006

Related U.S. Application Data
(60) Provisional application No. 60/516,596, filed on Oct. 31, 2003.

(30) Foreign Application Priority Data
Oct. 30, 2003 (DK) PA 2003 01612

(51) Int. Cl. A61K 39/385; A61K 38/21; C07H 21/04; C07K 14/475; C12N 1/15; C12N 1/21; C12N 15/63; C12N 15/74; C12N 15/79; C12N 5/04; C12N 5/06

(52) U.S. Cl. 424/85.2; 424/185.1; 424/192.1; 424/195.11; 424/198.1; 424/85.4; 435/243; 435/252.3; 435/252.31; 435/252.33; 435/254.2; 435/258.1; 435/320.1; 435/325; 435/348; 435/419; 435/471; 435/474; 435/476; 530/403; 536/23.5

(57) ABSTRACT

The present invention provides for novel immunogenic variants of VEGF (vascular endothelial growth factor) which are useful in active specific immunotherapy against diseases that are characterized by overexpression of VEGF. The invention also relates to methods of treating such diseases (for instance cancer) as well as to various tools in molecular biology that assist in the provision of the immunogenic variants.

### FIELD OF THE INVENTION

The present invention relates to the field of therapeutic immunotherapy, and in particular to the field of active immunotherapy targeted at down-regulating the autologous (“self”) protein Vascular Epithelial Growth Factor, VEGF. The invention thus provides novel and improved immunogenic variants of this dimeric protein as well as the necessary tools for the preparation of such variants. The invention further relates to methods of immunotherapy and anti-cancer therapy as well as compositions useful in such methods.

### BACKGROUND OF THE INVENTION

Vascular Epithelial Growth Factor (“VEGF”, also referred to as “VEGFA” and “VEGF-A”) is a naturally occurring protein in the body whose normal role is to trigger angiogenesis. In healthy adults, the need for active angiogenesis is limited to wound healing, endometrial proliferation, postlactational mammary gland involution and pregnancy. By contrast, angiogenesis is crucial for the growth and metastasis of tumors.

Angiogenesis requires stimulation of vascular endothelial cells that occurs via the release of angiogenic peptides, of which VEGF is the most potent. VEGF stimulates a number of biological events, including endothelial cell mitogenesis and migration; induction of proteases leading to re-modelling of the extracellular matrix; Increased vascular permeability and vasodilatation; immune modulation via inhibition of antigen-presenting cells; and maintenance of survival for newly formed blood vessels by inhibiting endothelial cell apoptosis. In colorectal cancer, increased VEGF expression correlates with invasiveness, vascular density, metastasis, recurrence, and prognosis [2, 3]. Blocking tumor-associated angiogenesis via VEGF neutralization has been shown to prevent tumor growth in a variety of animal models and anti-VEGF therapeutic activity was recently confirmed by phase II and III clinical data in CRC [4, 5].

The biological activities of VEGF are mediated through binding to one of three endothelial cell surface receptors: VEGF-R1 (flt-1), VEGF-R2 (flk-1/KDR), and VEGF-R3. Of these, the VEGF-R2 has a more restricted expression on endothelial cells and is upregulated once these cells proliferate during angiogenesis, such as during neovascularization of tumors. VEGF-R2 is strongly implicated as a therapeutic target and several approaches have been used to block VEGF-R2, including monoclonal antibody [6, 7], vaccines [8, 9], and synthetic receptor tyrosine kinase inhibitors [10], which have all demonstrated anti-tumor effects in several animal models.

Clinical and pre-clinical data obtained with anti-VEGF and anti-VEGF-R2 therapies clearly designate the VEGF/VEGF-R2 system as a biological pathway crucial to tumor survival and metastasis, and therefore as a relevant therapeutic target.

Use of active immunotherapy (“vaccination”) as a means of curing or alleviating disease has received growing attention over the last 2 decades. Notably, the use of active immunotherapy as a means for breaking tolerance to autologous proteins that are somehow related to a pathological (or otherwise undesired) physiologic condition has been known since the late seventies where the first experiments with antifertility vaccines where reported.

Vaccines against autologous antigens have traditionally been prepared by “immunogenizing” the relevant self-protein, e.g. by chemical coupling (“conjugation”) to a large foreign and immunogenic carrier protein (cf. U.S. Pat. No. 4,161,519) or by preparation of fusion constructs between the autologous protein and the foreign carrier protein (cf. WO 86/07383). In such constructs, the carrier part of the immunogenic molecule is responsible for the provision epitopes for T-helper lymphocytes (“T_{H} epitopes”) that render possible the breaking of autotolerance.

Later research has proven that although such strategies may indeed provide for the breaking of tolerance against autologous proteins, a number of problems are encountered. Most important is the fact that the immune response that is induced over time will be dominated by the antibodies directed against the carrier portion of the immunogen whereas the reactivity against the autologous protein often declines, an effect that is particularly pronounced when the carrier has previously served as an immunogen—this phenomenon is known as carrier suppression (cf. e.g. Kaliyaperumal et al. 1995., Eur. J. Immunol 25, 3375-3380). However, when using therapeutic vaccination it is usually necessary to re-immunize several times per year and to maintain this treatment for a number of years and this also results in a situation where the immune response against the carrier portion will be Increasingly dominant on the expense of the immune response against the autologous molecule.

Further problems involved when using hapten-carrier technology for breaking autotolerance is the negative steric effects exerted by carrier on the autologous protein part in such constructs: The number of accessible B-cell epitopes that resemble the conformational patterns seen in the native autologous protein is often reduced due to simple shielding or masking of epitopes or due to conformational changes induced in the self-part of the immunogen. Finally, it is very often difficult to characterize a hapten-carrier molecule in sufficient detail.

WO 95/05849 provided for a refinement of the above-mentioned hapten-carrier strategies. It was demonstrated that self-proteins wherein is in-substituted as little as one single foreign T_{H} epitope are capable of breaking tolerance towards the autologous protein. Focus was put on the preservation of tertiary structure of the autologous protein in order to ensure that a maximum number of autologous B-cell epitopes would be preserved in the immunogen in spite of the introduction of the foreign T_{H} element. This strategy has generally proven extremely successful inasmuch as the antibodies induced are broad-spectred as well as of high affinity and that the immune response has an earlier onset and a higher titer than that seen when immunizing with a traditional carrier construct.

WO 00/20027 provided for an expansion of the above principle. It was found that introduction of single T_{H} epitopes in the coding sequence for self-proteins could induce cytotoxic T-lymphocytes (CTLs) that reacts specifically with cells expressing the self-protein. The technology of WO 00/20027 also provided for combined therapy, where both antibodies and CTLs are induced—In these embodiments, the immunogens would still be required to preserve a substantial fraction of B-cell epitopes.

### OBJECT OF THE INVENTION

It is an object of the Invention to provide for immunogenic analogues of VEGF as well as to provide for methods for inducing humoral immunity against this protein, notably in the treatment of solid tumours. Finally, it is also objects of the invention to provide for means and measures that are useful when preparing or utilising the immunogens.

### SUMMARY OF THE INVENTION

The vascular epidermal growth factor (VEGF) and VEGF receptor (VEGFR) system plays a crucial role in regulating the process of normal as well as pathological angiogenesis, the formation of new blood vessels. As invasion and metastasis of all solid tumors rely on angiogenesis to nourish the tumor, the VEGF/VEGFR system provides an attractive target for therapeutic intervention.

Recent phase III clinical data with a monoclonal antibody targeting VEGF, (Avastin™/bevacizumab, Genentech) demonstrated that anti-VEGF antibodies combined with standard-of-care chemotherapy markedly extends survival of metastatic colorectal cancer (CRC) patients [1].

The clinical data clearly indicate that neutralizing VEGF activity, by infusion of monoclonal antibodies, can have positive effects in patients with solid tumors.

The present inventors have devised an attractive alternative, i.e. to harness the patient's own immune system to produce antibodies to neutralize VEGF via a vaccine approach that bypasses immunological tolerance and can be used to generate neutralizing antibodies to self-proteins like VEGF. This is achieved by active immunization with recombinant VEGF proteins modified to contain a highly immunodominant and promiscuous foreign peptide recognized by T helper cells. Due to functional tolerance, only T helper cells that recognize the inserted foreign epitope become activated. These activated T helper cells can then provide the necessary signals for VEGF-specific B cells to differentiate into antibody-secreting plasma cells. The antibodies produced by these plasma cells are then capable of neutralizing or clearing VEGF in vivo. In general terms, this process is inherently similar to any normal immune response driven by T cells responding to foreign antigens. The present approach simply harnesses these foreign-specific T helper cells to drive the anti-VEGF immune response. Importantly, In the absence of this foreign T helper response the anti-VEGF immune response wanes.

In its broadest and most general scope, the invention relates to a method for in vivo down-regulation of Vascular Endothelial Growth Factor (VEGF) activity in an animal, including a human being, the method comprising effecting presentation to the animal's immune system of an immunogenically effective amount of
at least one autologous VEGF protein or an autologous VEGF polypeptide or subsequence thereof which has been formulated so that immunization of the animal with the VEGF protein or VEGF polypeptide or subsequence thereof induces production of antibodies the animal's autologous VEGF protein, and/or
at least one VEGF analogue, which comprises a VEGF polypeptide wherein is introduced at least one modification in the VEGF amino acid sequence which has as a result that immunization of the animal with the analogue induces production of antibodies against the animal's autologous VEGF protein.

The invention further provides for nucleic acid fragments (such as DNA fragments) encoding such immunogenic analogues and also to vectors including such DNA fragments.

The invention also provides for transformed cells useful for preparing the analogues.

The invention further provides for immunogenic compositions comprising the analogous or the vectors of the invention.

Also provided by the invention are methods of treatment, where VEGF is down-regulated and to treatment of specific diseases, such as malignant neoplasms, where VEGF has an important role In angiogenesis and thereby in severity of the disease.

### DETAILED DISCLOSURE OF THE INVENTION

Definitions

In the following, a number of terms used in the present specification and claims will be defined and explained in detail in order to clarify the metes and bounds of the invention.

The terms “T-lymphocyte” and “T-cell” will be used interchangeably for lymphocytes of thymic origin that are responsible for various cell mediated immune responses as well as for helper activity in the humeral immune response. Likewise, the terms “B-lymphocyte” and “B-cell” will be used Interchangeably for antibody-producing lymphocytes.

“An immunogenic analogue” (or an “immunogenized” analogue or variant) is herein meant to designate a single polypeptide or protein that includes substantial parts of the sequence information found in native VEGF.

A “monomerized” analogue or variant of VEGF is in the present context a single polypeptide that includes, in covalently linked form via a peptide bond, the 2 polypeptide chains found in a polymeric protein in nature, where these 2 polypeptide chains are not linked via a peptide bond.

“A substantial fragment” of VEGF is intended to mean a part of a VEGF polypeptide that constitutes at least enough of the monomeric VEGF polypeptide so as to form a domain that folds up in substantially the same 3D conformation as can be found in the dimeric protein.

A “VEGF protein” is a functional VEGF found in vivo, i.e. in humans a VEGF protein is a homodimer.

A “VEGF polypeptide” is herein intended to denote single-chain polypeptides having an amino acid sequence derived from VEGF proteins from humans or other mammals. Unglycosylated forms of VEGF, which are prepared in prokaryotic system are included within the boundaries of the term as are forms having varying glycosylation patterns due to the use of e.g. yeasts or other non-mammalian eukaryotic expression systems. It should, however, be noted that when using the term “a VEGF polypeptide” it is intended that the polypeptide in question is normally non-immunogenic when presented to the animal to be treated. In other words, the VEGF polypeptide is a self-molecule or is a xeno-analogue of such a self-molecule which will not normally give rise to an immune response against VEGF of the animal in question.

A “VEGF analogue” is a molecule that includes a VEGF polypeptide which has been either subjected to changes in its primary structure and/or that is associated with elements from other molecular species. Such a change can e.g. be in the form of fusion of a VEGF polypeptide to a suitable fusion partner (i.e. a change in primary structure exclusively involving C- and/or N-terminal additions of amino acid residues) and/or it can be in the form of insertions and/or deletions and/or substitutions in the VEGF polypeptide's amino acid sequence. Also encompassed by the term are derivatized VEGF molecules, cf. the discussion below of modifications of VEGF.

It will be understood, that VEGF analogues also include monomeric variants that contains substantial parts of a complete dimeric VEGF protein.

When using the abbreviation “VEGF” herein, this is intended as references to the amino acid sequence of a mature, wildtype VEGF (also denoted “VEGFm” and “VEGFwt”. Mature huma VEGF is denoted hVEGF, hVEGFm or hVEGFwt, and murine mature VEGF is denoted mVEGF, mVEGFm, or mVEGFwt. In cases where a DNA construct includes information encoding a leader sequence or other material, this will be clear from the context.

The term “polypeptide” is in the present context intended to mean both short peptides of from 2 to 10 amino acid residues, oligopeptides of from 11 to 100 amino acid residues, and polypeptides of more than 100 amino acid residues. Furthermore, the term is also Intended to include proteins, i.e. functional biomolecules comprising at least one polypeptide; when comprising at least two polypeptides, these may form complexes, be covalently linked, or may be non-covalently linked. The polypeptide(s) in a protein can be glycosylated and/or lipidated and/or comprise prosthetic groups.

The term “subsequence” means any consecutive stretch of at least 3 amino acids or, when relevant, of at least 3 nucleotides, derived directly from a naturally occurring VEGF amino acid sequence or nucleic acid sequence, respectively.

The term “animal” is in the present context in general intended to denote an animal species (preferably mammalian), such as Homo sapiens, Canis domesticus, etc. and not just one single animal. However, the term also denotes a population of such an animal species, since it is important that the individuals immunized according to the method of the invention all harbour substantially the same VEGF allowing for immunization of the animals with the same immunogen(s). If, for instance, genetic variants of VEGF exist in different human populations it may be necessary to use different immunogens in these different populations in order to be able to break the autotolerance towards VEGF in each population. It will be clear to the skilled person that an animal in the present context is a living being which has an immune system. It is preferred that the animal is a vertebrate, such as a mammal.

By the term “down-regulation” is herein meant reduction in the living organism of the biological activity of VEGF (e.g. by interference with the interaction between VEGF and biologically important binding partners for this molecule). The down-regulation can be obtained by means of several mechanisms: Of these, simple interference with the active site in VEGF by antibody binding is the most simple. However, it is also within the scope of the present invention that the antibody binding results in removal of VEGF by scavenger cells (such as macrophages and other phagocytic cells).

The expression “effecting presentation . . . to the immune system” is intended to denote that the animal's immune system is subjected to an immunogenic challenge in a controlled manner. As will appear from the disclosure below, such challenge of the immune system can be effected in a number of ways of which the most Important are vaccination with polypeptide containing “pharmaccines” (i.e. a vaccine which is administered to treat or ameliorate ongoing disease) or nucleic acid “pharmaccine” vaccination. The important result to achieve is that immune competent cells in the animal are confronted with the antigen in an immunologically effective manner, whereas the precise mode of achieving this result is of less importance to the inventive idea underlying the present invention.

The term “immunogenically effective amount” has its usual meaning in the art, i.e. an amount of an immunogen which is capable of inducing an immune response which significantly engages pathogenic agents which share immunological features with the immunogen.

When using the expression that the VEGF has been “modified” is herein meant a chemical modification of the polypeptide which constitutes the backbone of VEGF. Such a modification can e.g. be derivatization (e.g. alkylation, acylation, esterification etc.) of certain amino acid residues In the amino acid sequence, but as will be appreciated from the disclosure below, the preferred modifications comprise changes of (or additions to) the primary structure of the amino acid sequence.

When discussing “autotolerance towards VEGF” it is understood that since VEGF is a self-protein in the population to be vaccinated, normal individuals in the population do not mount an immune response against it; it cannot be excluded, though, that occasional individuals in an animal population might be able to produce antibodies against native VEGF, e.g. as part of an autoimmune disorder. At any rate, an animal species will normally only be autotolerant towards its own VEGF, but it cannot be excluded that analogues derived from other animal species or from a population having a different phenotype would also be tolerated by said animal.

A “foreign T-cell epitope” (or: “foreign T-lymphocyte epitope”) is a peptide which is able to bind to an MHC molecule and which stimulates T-cells in an animal species—an alternate term is therefore. Preferred foreign T-cell epitopes in the invention are “promiscuous” (or “universal” or “broad-rangen”) epitopes, i.e. epitopes that bind to a substantial fraction of a particular class of MHC molecules in an animal species or population. Only a very limited number of such promiscuous T-cell epitopes are known, and they will be discussed in detail below. It should be noted that in order for the immunogens which are used according to the present invention to be effective in as large a fraction of an animal population as possible, it may be necessary to 1) insert several foreign T-cell epitopes in the same analogue or 2) prepare several analogues wherein each analogue has a different promiscuous epitope inserted. It should be noted also that the concept of foreign T-cell epitopes also encompasses use of cryptic T-cell epitopes, i.e. epitopes which are derived from a self-protein and which only exerts immunogenic behaviour when existing in isolated form without being part of the self-protein in question.

A “foreign T helper lymphocyte epitope” (a foreign T_{H} epitope) is a foreign T cell epitope which binds an MHC Class II molecule and can be presented on the surface of an antigen presenting cell (APC) bound to the MHC Class II molecule.

An “MHC Class II binding amino acid sequence that is heterologous to VEGF” is therefore an MHC Class II binding peptide that does not exist in VEGF. Such a peptide will, if it is also truly foreign to the animal species harbouring VEGF, be a foreign T_{H} epitope.

A “functional part” of a (bio)molecule is in the present context intended to mean the part of the molecule which is responsible for at least one of the biochemical or physiological effects exerted by the molecule. It is well-known in the art that many enzymes and other effector molecules have an active site which is responsible for the effects exerted by the molecule in question. Other parts of the molecule may serve a stabilizing or solubility enhancing purpose and can therefore be left out if these purposes are not of relevance in the context of a certain embodiment of the present invention. However, according to the present invention, it is preferred to utilise as much of the polymeric molecule as possible, because the increased stability has in fact been demonstrated when using the monomers described herein.

The term “adjuvant” has its usual meaning in the art of vaccine technology, i.e. a substance or a composition of matter which is 1) not in itself capable of mounting a specific immune response against the immunogen of the vaccine, but which is 2) nevertheless capable of enhancing the immune response against the immunogen. Or, in other words, vaccination with the adjuvant alone does not provide an immune response against the immunogen, vaccination with the immunogen may or may not give rise to an immune response against the immunogen, but the combination of vaccination with immunogen and adjuvant induces an immune response against the immunogen which is stronger than that induced by the immunogen alone.

“Targeting” of a molecule is in the present context intended to denote the situation where a molecule upon introduction in the animal will appear preferentially in certain tissue(s) or will be preferentially associated with certain cells or cell types. The effect can be accomplished in a number of ways including formulation of the molecule in composition facilitating targeting or by introduction in the molecule of groups which facilitates targeting. These issues will be discussed in detail below.

“Stimulation of the immune system” means that a substance or composition of matter exhibits a general, non-specific immunostimulatory effect. A number of adjuvants and putative adjuvants (such as certain cytokines) share the ability to stimulate the immune system. The result of using an immunostimulating agent is an increased “alertness” of the immune system meaning that simultaneous or subsequent immunization with an immunogen induces a significantly more effective immune response compared to isolated use of the immunogen.

Characteristics of the Immunogenic VEGF Analogues Used in the Invention

Although possible, it is not preferred to immunize with complete VEGF polypeptides or proteins or simple fragments thereof, since this will require formulation with strong adjuvants in order to induce an anti-self VEGF immune response. Rather, it is preferred to use an analogue of VEGF where at least one modification is present in the VEGF amino acid sequence. The modification can have the effect that at least one foreign T helper lymphocyte epitope (T_{H} epitope) is introduced, and/or that at least one first moiety is introduced which effects targeting of the modified molecule to an antigen presenting cell (APC) or a B-lymphocyte, and/or that at least one second moiety is introduced which stimulates the immune system, and/or that at least one third moiety is introduced which optimizes presentation of the modified VEGF polypeptide to the immune system.

Thus, the modification may introduction as side groups, by covalent or non-covalent binding to suitable chemical groups in the VEGF polypeptide or a subsequence thereof, of the foreign T_{H} epitope and/or of the first and/or of the second and/or of the third moiety, meaning that the moieties or the T_{H} epitope are fused to or otherwise coupled to or introduced into the VEDF polypeptide chain.

Targeting moieties are conveniently selected from the group consisting of a substantially specific binding partner for a B-lymphocyte specific surface antigen or for an APC specific surface antigen, such as a hapten or a carbohydrate for which there is a receptor on the B-lymphocyte or the APC. The immune stimulating moieties may be selected from the group consisting of a cytokine, a hormone, and a heat-shock protein. The presentation optimising moiety may be selected from the group consisting of a lipid group, such as a palmitoyl group, a myristyl group, a farnesyl group, a geranyl-geranyl group, a GPI-anchor, and an N-acyl diglyceride group.

A suitable cytokine is, or is an effective part of any of, interferon γ (IFN-γ), Flt3L, interleukin 1 (IL-1), interleukin 2 (IL-2), interleukin 4 (IL-4), interleukin 6 (IL-6), interleukin 12 (IL-12), interleukin 13 (IL-13), interleukin 15 (IL-15), and granulocyte-macrophage colony stimulating factor (GM-CSF), and the heat-shock protein is selected from, or is an effective part of any of, HSP70 (heat shock protein 70), HSP90, HSC70 (heat shock cognate 70), GRP94, and calreticulin (CRT).

A preferred heat-shock protein is, or is an effective part of any of, HSP70, HSP90, HSC70, GRP94, and calreticulin (CRT).

Other sequence changes that may enhance immunogenicity is duplication of at least one VEGF B-cell epitope and/or Introduction of a hapten.

Introduction of the moieties or of the foreigh T_{H} epitopes may include amino acid substitution and/or deletion and/or insertion and/or addition, the latter option providing for a fusion polypeptide.

It is preferred that introduction of the amino acid substitution and/or deletion and/or insertion and/or addition results in a substantial preservation of the overall tertiary structure of the VEGF polypeptide and since VEGF is a dimeric protein, it is also preferred that introduction of the amino acid substitution and/or deletion and/or insertion and/or addition results in a substantial preservation of the overall quarternary structure of the autologous VEGF protein. Both these restraints entail that the 3D structure of VEGF is essentially preserved.

It is namely advantageous if the immunogenic analogue according to the Invention displays, in the substantial fragments, a substantial fraction of B-cell epitopes found in the corresponding VEGF monomers when being part of the dimeric protein. A substantial fraction of B-cell epitopes is herein intended to mean a fraction of B-cell epitopes that antigenically characterises the dimeric protein versus other proteins and this is best accomplished when the immunogenic analogue is as close in 3D structure to the original native protein as possible.

It is preferred that the analogue displays essentially all B-cell epitopes found in the corresponding monomers when being part of the dimeric VEGF—of course, introduction of minor changes in the monomer sequence may be necessary. For instance may an amino acid sequence derived from a monomeric unit be modified by means of amino acid insertion, substitution, deletion or addition so as to reduce toxicity/physiological activity of the analogue as compared to the dimeric protein and/or so as to introduce the MHC Class II binding amino acid sequence.

An especially preferred embodiment provides for an immunogenic analogue of the invention, comprising essentially the complete amino acid sequence of each monomeric VEGF unit, either as a continuous sequence or as a sequence including inserts. That is, only insignificant parts of the monomeric unit's sequence are left out of the analogue, e.g. in cases where such a sequence does not contribute to tertiary structure of the monomeric unit or quarternary structure of the dimeric protein. However, this embodiment allows for substitution or insertion of the monomer, as long as the 3D structure of the dimeric protein is maintained. Hence, it is especially advantageous if the immunogenic analogue is one, wherein amino acid sequences of both units of the dimeric VEGF protein are represented in the analogue, and it is particularly advantageous if the analogue includes the complete amino acid sequences of both the monomers, either as unbroken sequences or as sequences including inserts.

As will appear, it is therefore preferred that the 3-dimensional structure of the complete VEGF protein is essentially preserved in the analogue.

Demonstration of preservation of a substantial fraction of B-cell epitopes or even the 3-dimensional structure of a VEGF protein that is subjected to modification as described herein can be achieved in several ways. One is simply to prepare a polyclonal antiserum directed against native VEGF (e.g. an antiserum prepared in a rabbit) and thereafter use this antiserum as a test reagent (e.g. in a competitive ELISA) against the modified proteins which are produced. Modified versions (analogues) which react to the same extent with the antiserum as does the native VEGF must be regarded as having the same 3D structure as the native VEGF whereas analogues exhibiting a limited (but still significant and specific) reactivity with such an antiserum are regarded as having maintained a substantial fraction of the original B-cell epitopes.

Alternatively, a selection of monoclonal antibodies reactive with distinct epitopes on VEGF can be prepared and used as a test panel. This approach has the advantage of allowing 1) an epitope mapping of VEGF and 2) a mapping of the epitopes which are maintained in the analogues prepared.

Of course, a third approach would be to resolve the 3-dimensional structure of VEGF (cf. above) and compare this to the resolved three-dimensional structure of the analogues prepared. Three-dimensional structure can be resolved by the aid of X-ray diffraction studies and NMR-spectroscopy. Further information relating to the tertiary structure can to some extent be obtained from circular dichroism studies which have the advantage of merely requiring the polypeptide in pure form (whereas X-ray diffraction requires the provision of crystallized polypeptide and NMR requires the provision of isotopic variants of the polypeptide) in order to provide useful information about the tertiary structure of a given molecule. However, ultimately X-ray diffraction and/or NMR are necessary to obtain conclusive data since circular dichroism can only provide indirect evidence of correct 3-dimensional structure via information of secondary structure elements.

The immunogenic analogue of the invention may include a peptide linker that includes or contributes to the presence in the analogue of at least one MHC Class II binding amino acid sequence that is heterologous to the VEGF protein. This is particularly useful in those cases where it is undesired to alter the amino acid sequence corresponding to the monomeric units of VEGF. Alternatively, the peptide linker may be free of and not contributing to the presence of an MHC Class II binding amino acid sequence in the animal species from where the VEGF protein is derived; this can conveniently be done in cases where it is necessary to utilise a very short linker or where it is advantageous to e.g. detoxify a potentially toxic analogue by introducing the MHC Class II binding element in an active site.

Both these embodiments can be combined with introduction of point mutations that detoxify (or render inactive) the molecule if need be, cf. below.

In other embodiments, no peptide linker is included, and in these cases the introduction of an MHC Class II binding amino acid sequence is performed by means of insertion, addition, deletion or substation in the VEGF polypeptide sequence.

It is preferred that the MHC Class II binding amino acid sequence binds a majority of MHC Class II molecules from the animal species from where the VEGF protein has been derived, i.e. that the MHC Class II binding amino acid sequence is universal or promiscuous.

It is of course important that this sequence serves its purpose as a T helper cell epitope in the species for which the immunogen is Intended to serve as a vaccine constituent. There exists a number of naturally occurring “promiscuous” (or “universal”) T-cell epitopes which are active in a large proportion of individuals of an animal species or an animal population and these are preferably introduced in the vaccine, thereby reducing the need for a very large number of different analogues in the same vaccine. Hence, the at least one MHC Class II binding amino acid sequence is preferably selected from a natural T-cell epitope and an artificial MHC-II binding peptide sequence. Especially preferred sequences are a natural T-cell epitope is selected from a Tetanus toxoid epitope such as P2 (SEQ ID NO: 16) or P30 (SEQ ID NO: 17), a diphtheria toxoid epitope, an influenza virus hemagluttinin epitope, and a P. falciparum CS epitope.

Over the years a number of other promiscuous T-cell epitopes have been identified. Especially peptides capable of binding a large proportion of HLA-DR molecules encoded by the different HLA-DR alleles have been identified and these are all possible T-cell epitopes to be introduced in the analogues used according to the present invention. Cf. also the epitopes discussed in the following references which are hereby all incorporated by reference herein: WO 98/23635 (Frazer IH et al., assigned to The University of Queensland); Southwood S et. al, 1998, J. Immunol. 160: 3363-3373; Sinigaglia F et al., 1988, Nature 336: 778-780; Chicz RM et al., 1993, J. Exp. Med 178: 27-47; Hammer J et al., 1993, Cell 74: 197-203; and Falk K et al., 1994, Immunogenetics 39: 230-242. The latter reference also deals with HLA-DQ and -DP ligands. All epitopes listed in these 5 references are relevant as candidate natural epitopes to be used in the present invention, as are epitopes that share common motifs with these.

Alternatively, the epitope can be any artificial T-cell epitope which is capable of binding a large proportion of MHC Class II molecules. In this context the pan DR epitope peptides (“PADRE”) described in WO 95/07707 and in the corresponding paper Alexander J et al., 1994, immunity 1: 751-761 (both disclosures are incorporated by reference herein) are interesting candidates for epitopes to be used according to the present invention. It should be noted that the most effective PADRE peptides disclosed in these papers carry D-amino acids in the C- and N-termini in order to improve stability when administered. However, the present invention primarily aims at incorporating the relevant epitopes as part of the analogue which should then subsequently be broken down enzymatically inside the lysosomal compartment of APCs to allow subsequent presentation in the context of an MHC-II molecule and therefore it is not expedient to incorporate D-amino acids in the epitopes used in the present invention.

One especially preferred PADRE peptide is the one having the amino acid sequence AKFVAAWTLKAAA (SEQ ID NO: 18) or an immunologically effective subsequence thereof. This, and other epitopes having the same lack of MHC restriction are preferred T-cell epitopes which should be present in the analogues used in the inventive method. Such super-promiscuous epitopes will allow for the most simple embodiments of the invention wherein only one single modified VEGF is presented to the vaccinated animal's immune system.

As mentioned above, the introduction of a foreign T-cell epitope can be accomplished by introduction of at least one amino acid insertion, addition, deletion, or substitution. Of course, the normal situation will be the introduction of more than one change in the amino acid sequence (e.g. Insertion of or substitution by a complete T-cell epitope) but the important goal to reach is that the analogue, when processed by an antigen presenting cell (APC), will give rise to such a T-cell epitope being presented in context of an MCH Class II molecule on the surface of the APC. Thus, if the amino acid sequence of the monomeric unit in appropriate positions comprises a number of amino acid residues which can also be found in a foreign T_{H} epitope then the introduction of a foreign T_{H} epitope can be accomplished by providing the remaining amino acids of the foreign epitope by means of amino acid Insertion, addition, deletion and substitution. In such a situation, it is not necessary to Introduce a complete T_{H} epitope by insertion or substitution.

It is preferred that the number of amino acid Insertions, deletions, substitutions or additions is at least 2, such as 3, 4, 5, 6, 7, 8, 9, 10, 11, 12, 13, 14, 15, 16, 17, 18, 19, 20, 21, and 25 insertions, substitutions, additions or deletions. It is furthermore preferred that the number of amino acid Insertions, substitutions, additions or deletions is not in excess of 150, such as at most 100, at most 90, at most 80, and at most 70. It is especially preferred that the number of substitutions, insertions, deletions, or additions does not exceed 60, and in particular the number should not exceed 50 or even 40. Most preferred is a number of not more than 30. With respect to amino acid additions, it should be noted that these, when the resulting construct is in the form of a fusion polypeptide, is often considerably higher than 150.

Preferred embodiments of the invention includes modification by introducing at least one foreign immunodominant T_{H} epitope (=“foreign MHC Class II binding amino acid sequence”). It will be understood that the question of immune dominance of a T_{H} epitope depends on the animal species in question. As used herein, the term “immunodominance” simply refers to epitopes which in the vaccinated individual gives rise to a significant immune response, but it is a well-known fact that a T_{H} epitope which is immunodominant in one Individual is not necessarily immunodominant in another individual of the same species, even though it may be capable of binding MHC-II molecules in the latter individual.

An Important point is the issue of MHC restriction of T_{H} epitopes. In general, naturally occurring T_{H} epitopes are MHC restricted, i.e. a certain peptide constituting a T_{H} epitope will only bind effectively to a subset of MHC Class II molecules. This in turn has the effect that in most cases the use of one specific T_{H} epitope will result in a vaccine component which is effective in a fraction of the population only, and depending on the size of that fraction, it can be necessary to include more T_{H} epitopes in the same molecule, or alternatively prepare a multi-component vaccine wherein the components are variants which are distinguished from each other by the nature of the T_{H} epitope introduced.

If the MHC restriction of the T-cells used is completely unknown (for instance in a situation where the vaccinated animal has a poorly defined MHC composition), the fraction of the animal population covered by a specific vaccine composition can be determined by means of the following formula:

$${f}_{popoulation} = 1 - {\prod }_{i = 1}^{n} ( 1 - {p}_{i} ) \qquad ( I )$$

where pᵢ is the frequency in the population of responders to the i^{th} foreign T-cell epitope present in the vaccine composition, and n is the total number of foreign T-cell epitopes in the vaccine composition. Thus, a vaccine composition containing 3 foreign T-cell epitopes having response frequencies in the population of 0.8, 0.7, and 0.6, respectively, would give
   1−0.2×0.3×0.4=0.976
i.e. 97.6 percent of the population will statistically mount an MHC-II mediated response to the vaccine.

The above formula does not apply in situations where a more or less precise MHC restriction pattern of the peptides used is known. If, for instance a certain peptide only binds the human MHC-II molecules encoded by HLA-DR alleles DR1, DR3, DR5, and DR7, then the use of this peptide together with another peptide which binds the remaining MHC-II molecules encoded by HLA-DR alleles will accomplish 100% coverage in the population in question. Likewise, if the second peptide only binds DR3 and DR5, the addition of this peptide will not increase the coverage at all. If one bases the calculation of population response purely on MHC restriction of T-cell epitopes in the vaccine, the fraction of the population covered by a specific vaccine composition can be determined by means of the following formula:

$${f}_{population} = 1 - {\prod }_{j = 1}^{3} {( 1 - {φ}_{j} )}^{2} \qquad ( II )$$

wherein φⱼ is the sum of frequencies in the population of allelic haplotypes encoding MHC molecules which bind any one of the T-cell epitopes in the vaccine and which belong to the f^{th} of the 3 known HLA loci (DP, DR and DQ); in practice, it is first determined which MHC molecules will recognize each T-cell epitope in the vaccine and thereafter these MHC molecules are listed by type (DP, DR and DQ)—then, the individual frequencies of the different listed allelic haplotypes are summed for each type, thereby yielding φ₁, φ₂, and φ₃.

It may occur that the value pᵢ in formula I exceeds the corresponding theoretical value πᵢ:

$${π}_{i} = 1 - {\prod }_{j = 1}^{3} {( 1 - {v}_{j} )}^{2} \qquad ( III )$$

wherein νⱼ is the sum of frequencies in the population of allelic haplotypes encoding MHC molecules which bind the i^{th} T-cell epitope in the vaccine and which belong to the j^{th} of the 3 known HLA loci (DP, DR and DQ). This means that in 1−πᵢ of the population there is a frequency of responders of f_{residual}_{_{—}}ᵢ=(pᵢ−πᵢ)/(1−πᵢ). Therefore, formula II can be adjusted so as to yield formula V:

$${f}_{population} = 1 - {\prod }_{j = 1}^{3} {( 1 - {φ}_{j} )}^{2} + ( 1 - {\prod }_{i = 1}^{n} ( 1 - {f}_{residual\_i} ) ) \qquad ( IV )$$
where the term 1−f_{residual}_{_{—}}ⱼ is set to zero if negative. It should be noted that formula IV requires that all epitopes have been haplotype mapped against identical sets of haplotypes.

Therefore, when selecting T-cell epitopes to be introduced in the analogue of the invention, it is important to include all knowledge of the epitopes which is available: 1) The frequency of responders in the population to each epitope, 2) MHC restriction data, and 3) frequency in the population of the relevant haplotypes.

It should be noted that preferred analogues of the invention comprise modifications which results in a polypeptide that includes stretches having a sequence identity of at least 70% with the corresponding monomeric units of the VEGF protein or with subsequences thereof of at least 10 amino acids in length. Higher sequence identities are preferred, e.g. at least 75% or even at least 80% or 85%. The sequence identity for proteins and nucleic acids can be calculated as (N_{ref}−N_{dif})·100/N_{ref}, wherein N_{dif} is the total number of non-identical residues in the two sequences when aligned and wherein N_{ref} is the number of residues in one of the sequences. Hence, the DNA sequence AGTCAGTC will have a sequence identity of 75% with the sequence AATCAATC (N_{dif}=2 and N_{ref}=8).

Finally, in order to conclusively verify that an analogue of the invention is indeed effective as an immunogen, various tests may be performed in order to provide the necessary confirmation, cf. also the specifics set forth in the examples herein. In this context, reference is also made to the discussion of identification of useful IL5 analogues in WO 00/65058—this disclosure may be used for verification of the usefulness of a VEGF analogue subject to the present inventive technology.

For hVEGF it is expected that constructs that mimic the natural hVEGF dimer structure and at the same time include foreign T_{H} elements will provide superior results compared to constructs based on the monomeric structure. This can be accomplished in essentially two ways, one where the VEGF polyepeptide is capable of folding correctly into a dimer structure during fermentation or subsequent refolding or by preparing monomerized constructs that e.g. include two VEGF polypeptides of analogues.

It is preferred that the VEGF polypeptide is a human VEGF-A polypeptide, preferably VEGF-A isoform 121 (SEQ ID NO: 5) or isoform 165 (SEQ ID NO: 4).

As will appear from the examples, one preferred analogue is a human VEGF-A polypeptide modified within the N-terminal part proximal to the first beta-strand and/or within the C-terminus of the cystine knot domain and/or within the loop between beta-strands B3 and B4.

Thus, especially preferred constructs are those wherein the human VEGF-A polypeptide has been modified by insertion into, deletion in, addition to, or substitution of any one of amino acids 1-15 In any one of SEQ ID NOs: 2-8. This means that preferred constructs entail insertion after any one of amino acids 1, 2, 3, 4, 5, 6, 7, 8, 9, 10, 11, 12, 13, 14 and 15 and/or deletion or substitution of any one of amino acids 1, 2, 3, 4, 5, 6, 7, 8, 9, 10, 11, 12, 13, 14 and 15 in SEQ ID NOs.: 2-8.

In another embodiment, the human VEGF-A polypeptide has been modified by insertion into, deletion in, addition to, or substitution of any amino acid C-terminal to residue 105 in any one of SEQ ID NOs: 2-8. This means that preferred constructs entail insertion after any one of amino acids 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206 In any one of SEQ ID NOs: 2-8 (insofar as said amino acid exists in that sequence) and or deletion or substitution of any one of amino acids 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206 in any one of SEQ ID NOs: 2-8 (insofar as said amino acid exists in that sequence).

In yet another embodiment, the human VEGF-A polypeptide has been modified by insertion, deletion or substitution in any one of SEQ ID NOs: 2-8, residues 59-66. This means that preferred constructs entail Insertion after any one of amino acids 59, 60, 61, 62, 63, 64, 65, or 66 and/or deletion or substitution of any one of amino acids 59, 60, 61, 62, 63, 64, 65, or 66.

In embodiments that rely on the monomerization principle, the analogue comprises a structure selected from
VEGFₘ-X-VEGF, VEGF-Xₘ-VEGF, VEGF-X-VEGFₙ, VEGFₘ-Xₘ-VEGF, VEGFₘ-X-VEGFₙ, VEGF-Xₘ-VEGFₙ, and VEGFₘ-Xₘ-VEGFₙ, wherein VEGF is a VEGF polypeptide or subsequence thereof, X is an inert linker, VEGFₘ is a VEGF polypeptide or subsequence thereof that includes a modification constituting or contributing to the presence of the at least one foreign T helper epitope in the analogue, VEGFₙ is a VEGF polypeptide or subsequence thereof that includes a modification constituting or contributing to the presence of the at least one foreign T helper epitope in the analogue, and Xₘ is a peptide linker that includes or contributes to the presence of the at least one foreign T helper epitope in the analogue. Especially preferred are constructs where the analogue has the formula VEGF-Xₘ-VEGF.

It is especially preferred that VEGFₘ, Xₘ and VEGFₙ comprise the P2 and/or P30 epitopes of tetanus toxoid or comprises a PADRE, and X is a di-glycine linker. However X may be any non-immunogenic linker peptide that does not give rise to MHC Class II binding sequences.

Protein/Polypeptide Vaccination and Formulation

When effecting presentation of the analogues to an animal's immune system by means of administration thereof to the animal, the formulation of the polypeptide follows the principles generally acknowledged in the art.

Preparation of vaccines which contain peptide sequences as active ingredients is generally well understood in the art, as exemplified by U.S. Pat. No. 4,608,251; 4,601,903; 4,599,231; 4,599,230; 4,596,792; and 4,578,770, all incorporated herein by reference. Typically, such vaccines are prepared as injectables either as liquid solutions or suspensions; solid forms suitable for solution in, or suspension in, liquid prior to injection may also be prepared. The preparation may also be emulsified. The active immunogenic ingredient is often mixed with excipients which are pharmaceutically acceptable and compatible with the active ingredient. Suitable excipients are, for example, water, saline, dextrose, glycerol, ethanol, or the like, and combinations thereof. In addition, if desired, the vaccine may contain minor amounts of auxiliary substances such as wetting or emulsifying agents, pH buffering agents, or adjuvants which enhance the effectiveness of the vaccines; cf. the detailed discussion of adjuvants below.

The vaccines are conventionally administered parenterally, by injection, for example, either subcutaneously, intracutaneously, intradermally, subdermally or intramuscularly. Additional formulations which are suitable for other modes of administration include suppositories and, in some cases, oral, buccal, sublinqual, intraperitoneal, intravaginal, anal, epidural, spinal, and intracranial formulations. For suppositories, traditional binders and carriers may include, for example, polyalkalene glycols or triglycerides; such suppositories may be formed from mixtures containing the active ingredient in the range of 0.5% to 10%, preferably 1-2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10-95% of active ingredient, preferably 25-70%. For oral formulations, cholera toxin is an Interesting formulation partner (and also a possible conjugation partner).

The polypeptides may be formulated Into the vaccine as neutral or salt forms. Pharmaceutically acceptable salts include acid addition salts (formed with the free amino groups of the peptide) and which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The vaccines are administered in a manner compatible with the dosage formulation, and in such amount as will be therapeutically effective and immunogenic. The quantity to be administered depends on the subject to be treated, including, e.g., the capacity of the individual's immune system to mount an immune response, and the degree of protection desired. Suitable dosage ranges are of the order of several hundred micrograms active ingredient per vaccination with a preferred range from about 0.1 μg to 2,000 μg (even though higher amounts in the 1-10 mg range are contemplated), such as in the range from about 0.5 μg to 1,000 μg, preferably in the range from 1 μg to 500 μg and especially in the range from about 10 μg to 100 μg. Suitable regimens for initial administration and booster shots are also variable but are typified by an initial administration followed by subsequent inoculations or other administrations.

The manner of application may be varied widely. Any of the conventional methods for administration of a vaccine are applicable. These include oral application on a solid physiologically acceptable base or in a physiologically acceptable dispersion, parenterally, by injection or the like. The dosage of the vaccine will depend on the route of administration and will vary according to the age of the person to be vaccinated and the formulation of the antigen.

In order to enhance immunogenicity of a polypeptide construct of the invention, it can be ensured that presentation to the immune system is effected by having at least two copies of the VEGF polypeptide, the subsequence thereof or the modified VEGF polypeptide covalently of non-covalently linked to a carrier molecule capable of effecting presentation of multiple copies of antigenic determinants. Such carriers may be polysaccharides or any other polymer substance capable of presenting polypeptides.

Some of the analogues of the vaccine are sufficiently immunogenic in a vaccine, but for some of the others the immune response will be enhanced if the vaccine further comprises an adjuvant substance.

Various methods of achieving adjuvant effect for the vaccine are known. General principles and methods are detailed in “The Theory and Practical Application of Adjuvants”, 1995, Duncan E. S. Stewart-Tull (ed.), John Wiley & Sons Ltd, ISBN 0-471-95170-6, and also in “Vaccines: New Generation Immunological Adjuvants”, 1995, Gregoriadis G et al. (eds.), Plenum Press, New York, ISBN 0-306-45283-9, both of which are hereby incorporated by reference herein.

It is especially preferred to use an adjuvant which can be demonstrated to facilitate breaking of the autotolerance to autoantigens; in fact, this is essential in cases where unmodified VEGF is used as the active ingredient in the autovaccine. Non-limiting examples of suitable adjuvants are selected from the group consisting of an immune targeting adjuvant; an immune modulating adjuvant such as a toxin, a cytokine, and a mycobacterial derivative; an oil formulation; a polymer; a micelle forming adjuvant; a saponin; an immunostimulating complex matrix (ISCOM matrix); a particle; DDA; aluminium adjuvants; DNA adjuvants; γ-inulin; and an encapsulating adjuvant. In general it should be noted that the disclosures above which relate to compounds and agents useful as first, second and third moieties in the analogues also refer mutatis mutandis to their use in the adjuvant of a vaccine of the invention.

The application of adjuvants include use of agents such as aluminium hydroxide or phosphate (alum), commonly used as 0.05 to 0.1 percent solution in buffered saline, admixture with synthetic polymers of sugars (e.g. Carbopol®) used as 0.25 percent solution, aggregation of the protein in the vaccine by heat treatment with temperatures ranging between 70° to 101° C. for 30 second to 2 minute periods respectively and also aggregation by means of cross-linking agents are possible. Aggregation by reactivation with pepsin treated antibodies (Fab fragments) to albumin, mixture with bacterial cells such as C. parvum or endotoxins or lipopolysaccharide components of gram-negative bacteria, emulsion in physiologically acceptable oil vehicles such as mannide mono-oleate (Aracel A) or emulsion with 20 percent solution of a perfluorocarbon (Fluosol-DA) used as a block substitute may also be employed. Admixture with oils such as squalene and IFA is also preferred.

According to the invention DDA (dimethyldioctadecylammonium bromide) is an interesting candidate for an adjuvant as is DNA and γ-inulin, but also Freund's complete and incomplete adjuvants as well as quillaja saponins such as QuilA and QS21 are interesting as is RIBI. Further possibilities are monophosphoryl lipid A (MPL), the above mentioned C3 and C3d, and muramyl dipeptide (MDP).

Liposome formulations are also known to confer adjuvant effects, and therefore liposome adjuvants are preferred according to the invention.

Also immunostimulating complex matrix type (ISCOM® matrix) adjuvants are preferred choices according to the invention, especially since it has been shown that this type of adjuvants are capable of up-regulating MHC Class II expression by APCs. An ISCOM® matrix consists of (optionally fractionated) saponins (triterpenoids) from Quillaja saponaria, cholesterol, and phospholipid. When admixed with the immunogenic protein, the resulting particulate formulation is what is known as an ISCOM particle where the saponin constitutes 60-70% w/w, the cholesterol and phospholipid 10-15% w/w, and the protein 10-15% w/w. Details relating to composition and use of immunostimulating complexes can e.g. be found in the above-mentioned text-books dealing with adjuvants, but also Morein B et al., 1995, Clin. Immunother. 3: 461-475 as well as Barr IG and Mitchell GF, 1996, Immunol. and Cell Biol. 74: 8-25 (both incorporated by reference herein) provide useful instructions for the preparation of complete immunostimulating complexes.

Another highly interesting (and thus, preferred) possibility of achieving adjuvant effect is to employ the technique described in Gosselin et al., 1992 (which is hereby incorporated by reference herein). In brief, the presentation of a relevant antigen such as an antigen of the present invention can be enhanced by conjugating the antigen to antibodies (or antigen binding antibody fragments) against the Fcγ receptors on monocytes/macrophages. Especially conjugates between antigen and anti-FcγRI have been demonstrated to enhance immunogenicity for the purposes of vaccination.

Other possibilities involve the use of the targeting and immune modulating substances (i.a. cytokines) mentioned in the claims as moieties for the protein constructs. In this connection, also synthetic inducers of cytokines like poly I:C are possibilities.

Suitable mycobacterial derivatives are selected from the group consisting of muramyl dipeptide, complete Freund's adjuvant, RIBI, and a diester of trehalose such as TDM and TDE.

Suitable immune targeting adjuvants are selected from the group consisting of CD40 ligand and CD40 antibodies or specifically binding fragments thereof (cf. the discussion above), mannose, a Fab fragment, and CTLA-4.

Suitable polymer adjuvants are selected from the group consisting of a carbohydrate such as dextran, PEG, starch, mannan, and mannose; a plastic polymer such as; and latex such as latex beads.

Yet another interesting way of modulating an immune response is to include the immunogen (optionally together with adjuvants and pharmaceutically acceptable carriers and vehicles) in a “virtual lymph node” (VLN) (a proprietary medical device developed by ImmunoTherapy, Inc., 360 Lexington Avenue, New York, N.Y. 10017-6501). The VLN (a thin tubular device) mimics the structure and function of a lymph node. Insertion of a VLN under the skin creates a site of sterile inflammation with an upsurge of cytokines and chemokines. T- and B-cells as well as APCs rapidly respond to the danger signals, home to the inflamed site and accumulate inside the porous matrix of the VLN. It has been shown that the necessary antigen dose required to mount an immune response to an antigen is reduced when using the VLN and that immune protection conferred by vaccination using a VLN surpassed conventional immunization using Ribi as an adjuvant. The technology is i.a. described briefly in Gelber C et al., 1998, “Elicitation of Robust Cellular and Humoral immune Responses to Small Amounts of immunogens Using a Novel Medical Device Designated the Virtual Lymph Node”, in: “From the Laboratory to the Clinic, Book of Abstracts, Oct. 12^{th}-15^{th} 1998, Seascape Resort, Aptos, Calif.”.

It is expected that the vaccine should be administered at least once a year, such as at least 1, 2, 3, 4, 5, 6, and 12 times a year. More specifically, 1-12 times per year is expected, such as 1, 2, 3, 4, 5, 6, 7, 8, 9, 10, 11, or 12 times a year to an individual in need thereof. It has previously been shown that the memory immunity induced by the use of the preferred auto-vaccines according to the invention is not permanent, and therefor the immune system needs to be periodically challenged with the analogues.

Due to genetic variation, different individuals may react with immune responses of varying strength to the same polypeptide. Therefore, the vaccine according to the invention may comprise several different polypeptides in order to increase the immune response, cf. also the discussion above concerning the choice of foreign T-cell epitope introductions. The vaccine may comprise two or more polypeptides, where all of the polypeptides are as defined above.

The vaccine may consequently comprise 3-20 different analogues, such as 3-10 analogues. However, normally the number of analogues will be sought kept to a minimum such as 1 or 2 analogues.

Nucleic Acid Vaccination

As a very important alternative to classic administration of a peptide-based vaccine, the technology of nucleic acid vaccination (also known as “nucleic acid immunisation”, “genetic immunisation”, and “gene immunisation”) offers a number of attractive features.

First, in contrast to the traditional vaccine approach, nucleic acid vaccination does not require resource consuming large-scale production of the immunogenic agent (e.g. in the form of industrial scale fermentation of microorganisms producing proteins). Furthermore, there is no need to device purification and refolding schemes for the immunogen. And finally, since nucleic acid vaccination relies on the biochemical apparatus of the vaccinated Individual in order to produce the expression product of the nucleic acid introduced, the optimum posttranslational processing of the expression product is expected to occur; this is especially important in the case of autovaccination, since, as mentioned above, a significant fraction of the original B-cell epitopes of the polymer should be preserved in the modified molecule, and since B-cell epitopes in principle can be constituted by parts of any (bio)molecule (e.g. carbohydrate, lipid, protein etc.). Therefore, native glycosylation and lipidation patterns of the immunogen may very well be of importance for the overall immunogenicity and this is expected to be ensured by having the host producing the immunogen.

It should be noted that the enhanced expression levels observed with the presently disclosed analogues is very important for efficacy of DNA vaccination, since the in vivo expression level is one of the determining factors in the immunogenic efficacy of a DNA vaccine

Hence, a preferred embodiment of the invention comprises effecting presentation of the analogue of the invention to the immune system by introducing nucleic acid(s) encoding the analogue into the animal's cells and thereby obtaining in vivo expression by the cells of the nucleic acid(s) introduced.

In this embodiment, the introduced nucleic acid is preferably DNA which can be in the form of naked DNA, DNA formulated with charged or uncharged lipids, DNA formulated in liposomes, DNA Included in a viral vector, DNA formulated with a transfection-facilitating protein or polypeptide, DNA formulated with a targeting protein or polypeptide, DNA formulated with Calcium precipitating agents, DNA coupled to an inert carrier molecule, DNA encapsulated in a polymer, e.g. in PLGA (cf. the microencapsulation technology described in WO 98/31398) or in chitin or chitosan, and DNA formulated with an adjuvant. In this context it is noted that practically all considerations pertaining to the use of adjuvants in traditional vaccine formulation apply for the formulation of DNA vaccines. Hence, all disclosures herein which relate to use of adjuvants in the context of polypeptide based vaccines apply mutatis mutandis to their use in nucleic acid vaccination technology.

As for routes of administration and administration schemes of polypeptide based vaccines which have been detailed above, these are also applicable for the nucleic acid vaccines of the invention and all discussions above pertaining to routes of administration and administration schemes for polypeptides apply mutatis mutandis to nucleic acids. To this should be added that nucleic acid vaccines can suitably be administered intraveneously and intraarterially. Furthermore, it is well-known in the art that nucleic acid vaccines can be administered by use of a so-called gene gun, and hence also this and equivalent modes of administration are regarded as part of the present invention. Finally, also the use of a VLN in the administration of nucleic acids has been reported to yield good results, and therefore this particular mode of administration is particularly preferred.

Furthermore, the nucleic acid(s) used as an immunization agent can contain regions encoding the moieties specified in the claims, e.g. in the form of the immunomodulating substances described above such as the cytokines discussed as useful adjuvants. A preferred version of this embodiment encompasses having the coding region for the analogue and the coding region for the immunomodulator in different reading frames or at least under the control of different promoters. Thereby it is avoided that the analogue or epitope is produced as a fusion partner to the immunomodulator. Alternatively, two distinct nucleotide fragments can be used, but this is less preferred because of the advantage of ensured co-expression when having both coding regions included in the same molecule.

Accordingly, the invention also relates to a composition for inducing production of antibodies against VEGF, the composition comprising
a nucleic acid fragment or a vector of the invention (cf. the discussion of nucleic acids and vectors below), and
a pharmaceutically and immunologically acceptable vehicle and/or carrier and/or adjuvant as discussed above.

Under normal circumstances, the nucleic acid is introduced in the form of a vector wherein expression is under control of a viral promoter. For more detailed discussions of vectors and DNA fragments according to the invention, cf. the discussion below. Also, detailed disclosures relating to the formulation and use of nucleic acid vaccines are available, cf. Donnelly J J et al, 1997, Annu. Rev. Immunol. 15: 617-648 and Donnelly J J et al., 1997, Life Sciences 60: 163-172. Both of these references are incorporated by reference herein.

Live Vaccines

A third alternative for effecting presentation of the analogues of the invention to the immune system is the use of live vaccine technology. In live vaccination, presentation to the immune system is effected by administering, to the animal, a non-pathogenic microorganism that has been transformed with a nucleic acid fragment encoding an analogue of the invention or with a vector incorporating such a nucleic acid fragment. The non-pathogenic microorganism can be any suitable attenuated bacterial strain (attenuated by means of passaging or by means of removal of pathogenic expression products by recombinant DNA technology), e.g. Mycobacterium bovis BCG., non-pathogenic Streptococcus spp., E. coli, Salmonella spp., Vibrio cholerae, Shigella, etc. Reviews dealing with preparation of state-of-the-art live vaccines can e.g. be found in Saliou P, 1995, Rev. Prat. 45: 1492-1496 and Walker P D, 1992, Vaccine 10: 977-990, both incorporated by reference herein. For details about the nucleic acid fragments and vectors used in such live vaccines, cf. the discussion below.

As an alternative to bacterial live vaccines, the nucleic acid fragment of the invention discussed below can be incorporated in a non-virulent viral vaccine vector such as a vaccinia strain or any other suitable pox virus.

Normally, the non-pathogenic microorganism or virus is administered only once to the animal, but in certain cases it may be necessary to administer the microorganism more than once in a lifetime in order to maintain protective immunity. It is even contemplated that immunization schemes as those detailed above for polypeptide vaccination will be useful when using live or virus vaccines.

Alternatively, live or virus vaccination is combined with previous or subsequent polypeptide and/or nucleic acid vaccination. For instance, it is possible to effect primary immunization with a live or virus vaccine followed by subsequent booster immunizations using the polypeptide or nucleic acid approach.

The microorganism or virus can be transformed with nucleic acid(s) containing regions encoding the moieties mentioned above, e.g. in the form of the immunomodulating substances described above such as the cytokines discussed as useful adjuvants. A preferred version of this embodiment encompasses having the coding region for the analogue and the coding region for the immunomodulator in different reading frames or at least under the control of different promoters. Thereby it is avoided that the analogue or epitopes are produced as fusion partners to the immunomodulator. Alternatively, two distinct nucleotide fragments can be used as transforming agents. Of course, having the adjuvating moieties in the same reading frame can provide, as an expression product, an analogue of the invention, and such an embodiment is especially preferred according to the present invention.

Combination Treatment

One especially preferred mode of carrying out the invention involves the use of nucleic acid vaccination as the first (primary) immunization, followed by secondary (booster) immunizations with a polypeptide based vaccine or a live vaccine as described above.

Use of the Method of the Invention in Treatment of Specific Diseases

All solid tumors rely on angiogenesis for their growth and metastatic properties while normal vasculature is quiescent in healthy adults, with each endothelial cell dividing once every 10 years. Angiogenesis provides then an attractive therapeutic target for therapy of solid tumours and with a theoretically limited toxicity profile.

Combined with conventional cytotoxic agents, anti-VEGF antibody therapy demonstrated potent anti-tumor activity in different cancers Including colorectal, kidney and lung cancers [4, 11, 12].

Colorectal cancer (“CRC”) is the second leading cause of cancer death in the United States and the American Cancer Society (ACS) estimates diagnosis of 147,500 new cases in United States in 2003. Current treatments Include surgery, and chemotherapy is prescribed to metastatic cancer patients for whom the 5-year survival rate is as low as 9%. Anti-VEGF anti-bodies added to standard-of-care chemotherapy increased both time to disease progression and median survival in CRC patients [4]. The treatment was generally well tolerated with thrombosis being the most significant adverse event. Active immunization with VEGF variants of the present Invention combined with standard-of-care chemotherapy would represent a novel therapeutic opportunity for CRC patients.

Anti-VEGF antibody treatment also demonstrated anti-tumor activity in phase II clinical trials involving non-small cell lung cancer (NSCLC) patients [11]. With 171,900 new cases expected in 2003, lung cancer is the leading cause of cancer death in United States. The 5-year relative survival rate for all stages combined is only 15% and only 3% for metastatic cancer patients. Time to disease progression was increased in NSCLC patients receiving anti-VEGF antibodies compared to control. Noteworthy, cases of pulmonary haemorrhage were observed in the anti-VEGF antibody arms, particularly in patients with squamous cell carcinoma (SCC), who should be excluded from further clinical trials with anti-VEGF agents.

Beside solid tumors as those listed above, anti-VEGF therapies may also be effective for the treatment of haematological malignancies, as suggested by a number of preclinical studies [13, 14]. Several clinical trials are currently testing this hypothesis. Further, a number of tumours that are in principal benign are also attractive targets for anti-VEGF therapy. Inoperable intracranial tumours and other benign tumours that are difficult surgical targets (i.a. anglofibroma rhinopharynx).

Altogether, these findings demonstrate the broad applicability of VEGF immunotherapy among solid and haematological tumor indications.

Angiogenesis is also an essential biological process to the progression of several major diseases, including cancer, diabetes, and inflammation. Excessive vascularization can contribute to some cardiovascular pathologies, such as atherosclerosis. Thus, regarding indications different from solid and non-solid tumors, anti-VEGF therapy according to the present invention could be applied to diseases characterized by chronic inflammation and in which angiogenesis is thought to play a role in the pathogenesis of the disease, e.g., rheumatoid arthritis, psoriasis, psoriatic arthritis, ankylosing spondylitis, inflammatory bowel diseases, bronchial asthma. Finally, diabetes and diabetic retinopathy could also constitute therapeutic Indications for VEGF immunization therapy.

Compositions of the Invention The invention also pertains to compositions useful in exercising the method of the invention.

Hence, the invention also relates to an immunogenic composition comprising an immunogenically effective amount of an analogue defined above, said composition further comprising a pharmaceutically and immunologically acceptable diluent and/or vehicle and/or carrier and/or excipient and optionally an adjuvant. In other words, this part of the invention concerns formulations of analogues, essentially as described hereinabove. The choice of adjuvants, carriers, and vehicles is accordingly in line with what has been discussed above when referring to formulation of the analogues for peptide vaccination.

The analogues are prepared according to methods well-known in the art. Longer polypeptides are normally prepared by means of recombinant gene technology including introduction of a nucleic acid sequence encoding the analogue into a suitable vector, transformation of a suitable host cell with the vector, expression of the nucleic acid sequence (by culturing the host cell under appropriate conditions), recovery of the expression product from the host cells or their culture supernatant, and subsequent purification and optional further modification, e.g. refolding or derivatization. Details pertaining to the necessary tools are found below under the heading “Nucleic acid fragments and vectors of the invention” but also in the examples.

Shorter peptides are, when relevant, preferably prepared by means of the well-known techniques of solid- or liquid-phase peptide synthesis. However, recent advances in this technology has rendered possible the production of full-length polypeptides and proteins by these means, and therefore it is also within the scope of the present invention to prepare the long constructs by synthetic means.

Nucleic Acid Fragments and Vectors of the Invention

It will be appreciated from the above disclosure that modified polypeptides can be prepared by means of recombinant gene technology but also by means of chemical synthesis or semi-synthesis; the latter two options are especially relevant when the modification consists of or comprises coupling to protein carriers (such as KLH, diphtheria toxoid, tetanus toxoid, and BSA) and non-proteinaceous molecules such as carbohydrate polymers and of course also when the modification comprises addition of side chains or side groups to an polymer-derived peptide chain. These embodiments, are, as will be understood from the above, not the preferred ones.

For the purpose of recombinant gene technology, and of course also for the purpose of nucleic acid immunization, nucleic acid fragments encoding the analogues are important chemical products (as are their complementary sequences). Hence, an important part of the invention pertains to a nucleic acid fragment which encodes an analogue as described herein, i.e. a polymer derived artificial polymer polypeptide as described in detail above. The nucleic acid fragments of the invention are either DNA or RNA fragments.

The nucleic acid fragments of the Invention will normally be inserted in suitable vectors to form cloning or expression vectors carrying the nucleic acid fragments of the invention; such novel vectors are also part of the invention. Details concerning the construction of these vectors of the invention will be discussed in context of transformed cells and microorganisms below. The vectors can, depending on purpose and type of application, be in the form of plasmids, phages, cosmids, mini-chromosomes, or virus, but also naked DNA which is only expressed transiently in certain cells is an important vector (and may be useful in DNA vaccination). Preferred cloning and expression vectors of the invention are capable of autonomous replication, thereby enabling high copy-numbers for the purposes of high-level expression or high-level replication for subsequent cloning.

The general outline of a vector of the invention comprises the following features in the 5′→3′ direction and in operable linkage: a promoter for driving expression of the nucleic acid fragment of the invention, optionally a nucleic acid sequence encoding a leader peptide enabling secretion (to the extracellular phase or, where applicable, into the periplasma) of or integration into the membrane of the polypeptide fragment, the nucleic acid fragment of the invention, and optionally a nucleic acid sequence encoding a terminator. When operating with expression vectors in producer strains or cell-lines it is for the purposes of genetic stability of the transformed cell preferred that the vector when introduced into a host cell is integrated in the host cell genome. In contrast, when working with vectors to be used for effecting in vivo expression in an animal (i.e. when using the vector in DNA vaccination) it is for security reasons preferred that the vector is not incapable of being integrated in the host cell genome; typically, naked DNA or non-integrating viral vectors are used, the choices of which are well-known to the person skilled in the art.

The vectors of the invention are used to transform host cells to produce the modified VEGF polypeptide of the invention. Such transformed cells, which are also part of the invention, can be cultured cells or cell lines used for propagation of the nucleic acid fragments and vectors of the invention, or used for recombinant production of the modified polypeptides of the invention. Alternatively, the transformed cells can be suitable live vaccine strains wherein the nucleic acid fragment (one single or multiple copies) have been inserted so as to effect secretion or integration into the bacterial membrane or cell-wall of the modified VEGF.

Preferred transformed cells of the invention are microorganisms such as bacteria (such as the species Escherichia [e.g. E. coli], Bacillus [e.g. Bacillus subtilis], Salmonella, or Mycobacterium [preferably non-pathogenic, e.g. M. bovis BCG]), yeasts (such as Saccharomyces cerevisiae), and protozoans. Alternatively, the transformed cells are derived from a multicellular organism such as a fungus, an insect cell, a plant cell, or a mammalian cell. Most preferred are cells derived from a human being, cf. the discussion of cell lines and vectors below. Recent results have shown great promise in the use of a commercially available Drosophila melanogaster cell line (the Schneider 2 (S₂) cell line and vector system available from Invitrogen) for the recombinant production of VEGF analogues of the invention, and therefore this expression system is particularly preferred, and therefore this type of system is also a preferred embodiment of the invention in general.

For the purposes of cloning and/or optimized expression it is preferred that the transformed cell is capable of replicating the nucleic acid fragment of the invention. Cells expressing the nucleic fragment are preferred useful embodiments of the invention; they can be used for small-scale or large-scale preparation of the analogue or, in the case of non-pathogenic bacteria, as vaccine constituents in a live vaccine.

When producing the analogues of the invention by means of transformed cells, it is convenient, although far from essential, that the expression product is either exported out into the culture medium or carried on the surface of the transformed cell, since both of these options facilitate subsequent purification of the expression product.

When an effective producer cell has been identified it is preferred, on the basis thereof, to establish a stable cell line which carries the vector of the invention and which expresses the nucleic acid fragment encoding the modified VEGF. Preferably, this stable cell line secretes or carries the VEGF analogue of the invention, thereby facilitating purification thereof.

In general, plasmid vectors containing replicon and control sequences that are derived from species compatible with the host cell are used in connection with the hosts. The vector ordinarily carries a replication site, as well as marking sequences which are capable of providing phenotypic selection in transformed cells. For example, E. coil is typically transformed using pBR322, a plasmid derived from an E. coil species (see, e.g., Bolivar et al., 1977). The pBR322 plasmid contains genes for ampicillin and tetracycline resistance and thus provides easy means for identifying transformed cells. The pBR plasmid, or other microbial plasmid or phage must also contain, or be modified to contain, promoters that can be used by the prokaryotic microorganism for expression.

Those promoters most commonly used in prokaryotic recombinant DNA construction include the B-lactamase (penicillinase) and lactose promoter systems (Chang et al., 1978; Itakura et al., 1977; Goeddel et al., 1979) and a tryptophan (trp) promoter system (Goeddel et al., 1979; EP-A-0 036 776). While these are the most commonly used, other microbial promoters have been discovered and utilized, and details concerning their nucleotide sequences have been published, enabling a skilled worker to ligate them functionally with plasmid vectors (Siebwenlist et al., 1980). Certain genes from prokaryotes may be expressed efficiently in E. coli from their own promoter sequences, precluding the need for addition of another promoter by artificial means.

In addition to prokaryotes, eukaryotic microbes, such as yeast cultures may also be used, and here the promoter should be capable of driving expression. Saccharomyces cerevisiase, or common baker's yeast is the most commonly used among eukaryotic microorganisms, although a number of other strains are commonly available. For expression in Saccharomyces, the plasmid YRp7, for example, is commonly used (Stinchcomb et al., 1979; Kingsman et al., 1979; Tschemper et al., 1980). This plasmid already contains the trpl gene which provides a selection marker for a mutant strain of yeast lacking the ability to grow in tryptophan for example ATCC No. 44076 or PEP4-1 (Jones, 1977). The presence of the trpl lesion as a characteristic of the yeast host cell genome then provides an effective environment for detecting transformation by growth in the absence of tryptophan.

Suitable promoting sequences in yeast vectors include the promoters for 3-phosphoglycerate kinase (Hitzman et al., 1980) or other glycolytic enzymes (Hess et al., 1968; Holland et al., 1978), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. In constructing suitable expression plasmids, the termination sequences associated with these genes are also ligated into the expression vector 3′ of the sequence desired to be expressed to provide polyadenylation of the mRNA and termination.

Other promoters, which have the additional advantage of transcription controlled by growth conditions are the promoter region for alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization. Any plasmid vector containing a yeast-compatible promoter, origin of replication and termination sequences is suitable.

In addition to microorganisms, cultures of cells derived from multicellular organisms may also be used as hosts. In principle, any such cell culture is workable, whether from vertebrate or invertebrate culture. However, interest has been greatest in vertebrate cells, and propagation of vertebrate in culture (tissue culture) has become a routine procedure in recent years (Tissue Culture, 1973). Examples of such useful host cell lines are VERO and HeLa cells, Chinese hamster ovary (CHO) cell lines, and W138, BHK, COS-7 293, Spodoptera frugiperda (SF) cells (commercially available as complete expression systems from i.a. Protein Sciences, 1000 Research Parkway, Meriden, Conn. 06450, U.S.A. and from Invitrogen), and MDCK cell lines. In the present invention, an especially preferred cell line the insect cell line S₂, available from Invitrogen, PO Box 2312, 9704 CH Groningen, The Netherlands.

Expression vectors for such cells ordinarily include (if necessary) an origin of replication, a promoter located in front of the gene to be expressed, along with any necessary ribosome binding sites, RNA splice sites, polyadenylation site, and transcriptional terminator sequences.

For use in mammalian cells, the control functions on the expression vectors are often provided by viral material. For example, commonly used promoters are derived from polyoma, Adenovirus 2, and most frequently Simian Virus 40 (SV40) or cytomegalovirus (CMV). The early and late promoters of SV40 virus are particularly useful because both are obtained easily from the virus as a fragment which also contains the SV40 viral origin of replication (Fiers et al., 1978). Smaller or larger SV40 fragments may also be used, provided there is included the approximately 250 bp sequence extending from the HindIII site toward the BglI site located in the viral origin of replication. Further, it is also possible, and often desirable, to utilize promoter or control sequences normally associated with the desired gene sequence, provided such control sequences are compatible with the host cell systems.

An origin of replicatfon may be provided either by construction of the vector to include an exogenous origin, such as may be derived from SV40 or other viral (e.g., Polyoma, Adeno, VSV, BPV) or may be provided by the host cell chromosomal replication mechanism. If the vector is integrated into the host cell chromosome, the latter is often sufficient.

### EXAMPLE 1

### Strategy for the Molecular Design of VEGF Protein Variants

VEGF-A is the main angiogenic factor among the VEGF protein family and is therefore the primary target of anti-angiogenesis therapy. The human VEGF-A gene is organized as eight exons separated with seven introns. Alternative exon splicing of the VEGF-A mRNA generates four main VEGF-A isoforms, VEGF-A121 (SEQ ID NO: 5), VEGF-A165 (SEQ ID NO: 4), VEGF-A189 (SEQ ID NO: 3) and VEGF-A206 (SEQ ID NO: 2), having respectively 121, 165, 189 and 206 amino acids after cleavage of the signal peptide. VEGF-A-189 lacks the 3′-end of exon 6, VEGF-A-165 lacks exon 6 and VEGF-A-121 lacks exons 6 and 7. Three additional, less frequent splicing isoforms also exist: VEGF-A-183 (SEQ ID NO: 6), which lacks part of exon 6, VEGF-A-148 (SEQ ID NO: 7), which lacks the 3′-end of exon 7 and exon 8 and VEGF-A-145 (SEQ ID NO: 8), which lacks the 3′-end part of exon 6 and exon 7.

VEGF-A is a homodimeric member of the cystine knot family of proteins. The two monomers are covalently linked via two disulfide bridges (C51-C51 and C60-C60). The amino acid region 1-110 (amino acid numbering from the VEGF-A isoform 121, SEQ ID NO: 5) comprises the receptor binding domain, the structure of which was determined by crystallization and X-ray diffraction [16,17]. This domain exhibits a characteristic cystine knot domain fold and consists primarily of a central four-stranded sheet and the cystine knot motif, a ring formed by the disulfide bridges C57-C102, C61-C104 and C26-C68 (numbering from the VEGF-A isoform 121, SEQ ID NO: 5) at one end of the domain and of a short three-stranded beta-sheet at the other end of the domain. The structures of the VEGF-A receptor binding domain in complex with its receptor [18] or with a receptor blocking peptide [19] or with a neutralizing monoclonal antibody [20] have also been determined. They highlight the area and amino acids of VEGF-A that are necessary for receptor binding and consecutive signal transduction. VEGF-A isoforms 165, 189 and 206 contain an additional, structurally independent domain, C-terminal to the cystine knot domain. This additional domain exhibits heparin binding properties. The NMR-structure of the heparin binding domain of VEGF-A isoform 165 has been determined [21]. It represents a novel fold, consisting of two subdomains, each containing two disulfide bridges and a short two-stranded anti-parallel beta-sheet. The C-terminal sub-domain also contains a short alpha-helix.

The design of VEGF variants is based primarily on sequence analysis and alignment of the seven VEGF-A isoforms (cf. SEQ ID NOs: 2-8) and sequence alignment of the five human members of the VEGF protein family (SEQ ID NOs: 1 and 9-12). Design is also based on analysis of the 3D-structure of VEGF-A with the aim to conserve the overall structure of the cystine knot domain in the VEGF AutoVac protein.

Choice of T_{H}-epitope

At least three T_{H}-epitope containing peptides are used for the VEGF variant design; these T_{H}-epitope containing peptides can be used alone or in combination within one VEGF variant: the tetanus toxoid epitope P2 (SEQ ID NO: 16), the tetanus toxoid epitope P30 (SEQ ID NO: 17) or a synthetic epitope of the PanDr family (e.g. SEQ ID NO: 18).

Choice of a VEGF-A Isoform

The template for VEGF variant design is preferably VEGF-A isoform 121 (SEQ ID NO: 5) or isoform 165 (SEQ ID NO: 4), but the present design strategy is applicable to all naturally occurring VEGF-A isoforms, (produced by alternative splicing and/or by proteolytic cleavage) as well as to the recombinantly expressed forms described in Christinger et al. [22] and to any truncated form that can be produced by protease cleavage in vitro.

Choice of Insertion/Substitution Sites

Certain areas of native VEGF-A are believed to be superiorly suited for performing modifications for design of immunogenic variants of VEGF. It is for instance predicted that modifications within at least the N-terminal part of the protein before the first beta-strand, within the C-terminus of the cystine knot domain, i.e. after amino acid 136 (numbering from the N-terminal amino acid of the unprocessed VEGF-A precursor in SEQ ID NO: 1) as well as within the loop between beta-strands B3 and B4 will be most likely to produce the desired constructs and vaccination results. The main consideration for choosing these areas is the preservation in the variant of the tertiary and quaternary structure of the VEGF protein.

Insertion/Substitution within the N-terminus

Herein, the amino acid sequence 1-15 of SEQ ID NO: 5 (APMAEGGGQNHHEVV) present in all VEGF-A isoforms is defined as the N-terminal region. This N-terminal region is selected as a primary target for T_{H}-epitope insertion/substitution since this region is poorly conserved within the VEGF family and it is structurally highly flexible showing no defined secondary structure elements.

Insertion/Substitution within the C-terminus

The amino acid sequence 16-105 of all VEGF-A isoforms constitute the core of the cystine knot domain. Herein, the area C-terminal to amino acid 105 of all VEGF-A isoforms set forth in SEQ ID NOs: 2-8 is defined as the C-terminal area. This area is also a target for T_{H}-epitope insertion/substitution, since this region is very poorly conserved among the VEGF family members and since it is structurally independent from the cystine knot domain.

Insertion/Substitution within the B3-B4 Loop

The loop (i.e. amino acids 59-66 of all VEGF-A isoforms set forth in SEQ ID NOs: 2-8) between beta-strands B3 and B4 is a secondary target for T_{H}-epitope insertion/substitution. This loop is situated in the vicinity of the cystine knot, which is considered to increase local stability and rigidity in the tertiary structure. Therefore insertion/substitution within the B3-B4 loop might not be disruptive for the overall tertiary structure of the protein.

T_{H}-epitope as a Linker Between two Monomer Domains

Besides the substitution/insertion design strategy, it is also the intention to prepare VEGF protein variants by covalently linking two VEGF-A cystine knot domains with a peptide linker comprising a foreign T_{H}-epitope. The main consideration behind this strategy is that the native VEGF cystine knot domain spontaneously forms a dimer and that the C-terminus of one monomer of VEGF is in close proximity to the N-terminus of the second monomer in the 3D-structure of VEGF-A [16,17]. Thus a T_{H}-epitope may be accommodated as a linker between two monomers without disturbing folding. The T_{H}-epitope will be inserted within the region 105-121 of the N-terminal monomer and region 1-15 of the C-terminal monomer by insertion or partial substitution (numbering from VEGF-A isoform 121, SEQ ID NO: 5).

De-activation of VEGF Variant Molecules

The structure of VEGF is known from several studies, including complexes with both the VEGFR1 (Flt-1) receptor, monoclonal antibodies and neutralizing peptides [18-20, 23]. Furthermore a mutational analysis of the binding to the VEGFR2 (KDR) receptor that maps the kinase domain receptor-binding site has also been published [17]. The KDR receptor dominates the angiogenic response and is as such the primary activity to block. From the literature and interaction/distance analysis of the VEGFR113 VEGF complex structure (the structures 1FLT & 1QTY in the Protein data bank, Brookhaven), it is apparent that both these receptors bind overlapping regions of the distal parts of the elongated homodimer. The interaction includes both monomers in the binding, which is divalent with respect to the binding of the receptors, due to the symmetry of the homodimer.

Neutralization or reduction of activity would thus be obtained by point mutating amino acid residues in one ore more positions in these regions.

The preferred regions for VGFR2 neutralizing mutations according to Muller et al. [17] and a structural analysis are:

| | |
|---|---|
| Region | Specific, in priority order |

VEGFR2 Muller, 1997 [17]:

| | |
|---|---|
| F17-Q22 | F17 M18 Y21 Q22 |
| I46-E64 | I46 E64 I43 F47 |
| Q79-P85 | Q79 I83 P85 Q89 M94 |

VEGFR1 (45, 41) 1FLT & 1QTY Protein data bank Brookhaven

| | |
|---|---|
| F17-Y25 | M18 - Y25 - Q22 - Y21 |
| K48 | K48 - I46 |
| D63-I91 | D63 - G65 - L66 - Q79 - M81 - I83 - Q89 - I91 |
| R105-P106 | R105 - P106 |

VEGFR1 + VEGFR2 from above

Region
F17-Y25
I43-L66
Q79-M94
R105-P106

Underlined Points of Mutation are the Most Preferred.

The mutations according to Muller 1997 are all substations of the amino acid in question with an alanine, and considerable loss of interaction with the receptor has been observed using this type of mutation alone. However, in order to further diminish binding of the VEGF variant to the receptor, it is contemplated to substitute “non-conservatively”, meaning that an optimum substitution strategy will involve substitution of polar with non-polar amino acids and vice versa, substitution of positively charged amino acids with negatively charged amino acids and vice versa.

### EXAMPLE 2

### Protein Expression of VEGF Variants

Various VEGF-A isoforms have been expressed recombinantly in a number of different expression systems including E. coli [22], insect cells [24] and CHO cells [25, 26]. All three expression systems will be considered for the present application.

Expression of Immunogenic VEGF Variants in E. coli

A synthetic cDNA fragment encoding for the desired VEGF variant will be cloned into a suitable expression vector, e.g. pET28. The resulting plasmid will be transformed in a suitable E. coli expression strain, e.g. HMS174(DE3). For expression, cultures of the resulting E. coli strain will be prepared in a fermentor. Expression of the recombinant protein is initiated by addition of IPTG or lactose at a chosen time. Expression is monitored and the culture is stopped at a chosen time. Cells are then harvested and the culture medium is discarded.

Expression of Immunogenic VEGF Variants in Insect Cells

A polyclonal culture of S2 Drosophila melanogaster cells will be transfected with a pMT vector (DES® system, Invitrogen) containing the gene coding the relevant VEGF variant. The cells will in parallel be transfected with a plasmid carrying a gene conferring hygromycin resistance enabling the usage of hygromycin for selection of transfected cells.

A limited dilution technique is used for isolation of single cell clones and a Master Cell Bank (MCB) can be produced from the selected cell line.

One vial from the MCB is resuscitated in a T-flask and propagated in shake flasks containing ExCell420 media (JRH) at 25° C. to obtain enough biomass for the inoculation of a bioreactor. A total of 45×10⁹ cells is diluted into 3000 mL with ExCell 420 supplemented with 4 mM Glutamine, 0.1% Pluronic F68, and 0.5 mL/L PD30 antifoam. The 3000 mL are used to inoculate an Applikon bioreactor (7 L working volume) where the culture will grow for 3 days at 25° C., dO₂=50% (100%=air saturation), pH=6.5±0.1 (adjusted with 5% H₃PO₄ and 0.5 M NaOH), and stirred at 170 rpm.

This culture is then diluted with ExCell 420 supplemented with 4 mM Glutamine, 0.1% Pluronic F68, and 0.5 mL/L PD30 antifoam to a total cell concentration of 15×106 cells/mL and used for inoculation of a 15 L working volume Applikon Bioreactor maintaining 25° C., dO₂=50% (sparging with pure oxygen), pH=6.5±0.1 (adjusted with 5% H₃PO₄ and 0.5 M NaOH), and stirred at 142 rpm. The culture will continuously be diluted with ExCell 420 supplemented with 4 mM Glutamine and 0.1% Pluronic F68 until a total volume of 10 L is reached. The dilution rate will be adjusted daily to prevent the cell number to drop below 15×10⁶ cells/mL. PD30 antifoam is added manually to the culture to maintain a total concentration of 0.5 mL/L.

When filling is completed, perfusion is initiated at 1 RV/day (reactor volumes per day) using the BioSep cell (AppliSens) acoustic retention device to prevent cell loss with the removed media. At a cell concentration of 30×10⁶ cells/mL, the culture is induced by addition of a total of 2 μM CdCl₂ (10 mM stock) to the culture and to the medium reservoir.

The fermentation medium is then harvested, centrifuged to obtain a cell free supernatant, and filtrated through a PALL filter 0.8/0.22 μm. The resulting sterile supernatant is either stored at −80° C. until use or stored at 4° C. for up to one week.

Expression in CHO Cells or Another Mammalian Expression System

Standard CHO expression systems may be used for this purpose, cf. the description above concerning mammalian expression systems.

### EXAMPLE 3

### Protein Purification and Characterization of VEGF Variants

Protein Purification of Recombinant VEGF Variant Proteins Expressed in E. coli

These proteins are typically expressed as insoluble proteins in inclusion bodies in E. coli [22]. After harvest and disruption (by pressure in a cell disrupter) of the E. coli cells, the inclusion bodies are isolated by filtration or centrifugation. The inclusion bodies are then washed with a combination of detergents and denaturants in order to remove hydrophobic E. coli proteins, which often contaminate inclusion bodies. The washed inclusion bodies, containing primarily recombinant VEGF variant protein are then dissolved in a buffer (e.g. 20 mM Tris pH 7,5) containing a chaotropic agent (e.g 4-8 M urea or 2-6 M guanidine hydrochloride) in the presence of 20 mM dithiothreitol to achieve complete reduction of disulfide bridges. Refolding is then achieved by removal of the denaturant either by dilution or dialysis or any other suitable method known to the skilled person. The refolding buffer may contain a redox pair to create the necessary oxidising potential for correct formation of the disulfide bonds. The refolded protein is then purifed using a combination of chromatographic steps, e.g. ion-exchange, hydrophobic interaction, chelating-affinity and size exclusion. For the variant proteins based on the VEGF-A isoforms containing a heparin binding domain, heparin affinity chromatography may be used for purification [24].

Characterisation of the purified proteins will include identity tests (e.g. N-terminal sequencing, mass spectrometry, amino acid analysis, immunoblotting) and structural characterisation (e.g. circular dichroism and in vitro receptor binding assay).

Protein Purification of Recombinant VEGF Variants Expressed in Insect Cells or in a Mammalian Expression System

These proteins are expressed and secreted to the culture medium. Cells are removed from the culture by filtration or centrifugation and the recombinant VEGF variant protein is purified from the cell-free culture supernatant. Purification may include protein precipitation steps (e.g. acid precipitation or ammonium sulfate fractionation) and several chromatography steps, e.g. ion-exchange, hydrophobic interaction, chelating-affinity and size exclusion. For the variant proteins based on the VEGF-A isoforms containing a heparin binding domain, heparin affinity chromatography may be used for purification [24].

Characterisation of the purified proteins will include identity tests (e.g. N-terminal sequencing, mass spectrometry, amino acid analysis, immunoblotting) and structural characterisation (e.g. circular dichroism and in vitro receptor binding assay).

### EXAMPLE 4

### In Vitro Proof of Efficacy of VEGF Variants

Anti-VEGF Antibody Response in VEGF Variant Immunized Animals

Enzyme-Linked ImmunoSorbent Assay (ELISA

Immunocompetent mice are immunized once or several times with human or mouse VEGF variant molecules of the present invention formulated in an appropriate adjuvant. Blood samples are collected at different time-points and anti-VEGF antibodies in the sera are detected by enzyme-linked immunosorbent assay (ELISA). Briefly, wild type recombinant VEGF molecules (of human or mouse origin) are either directly coated onto ELISA plates or bound to coated anti-(human or mouse)VEGF antibodies. Serial dilutions of VEGF antiserum are added to the wells for measurement of VEGF-specific antibody titer.

ExtraCellular Matrix (ECM) Assay

Anti-VEGF antibodies produced in VEGF variant-immunized animals (as described in Example 5) are tested for their ability to recognize native VEGF molecules present in the ECM. Briefly, ECM-coated plates (obtained from a commercial source such as Becton Dickinson) are incubated with varying concentrations of VEGF antiserum. After washing steps, plates are incubated with alkaline-phosphatase-conjugated anti-mouse or anti-human Fc antibody. Reagent buffer is further added for colour development. This assay gives further indications on the avidity of the polyclonal VEGF antiserum generated after vaccination with VEGF variants.

Neutralizing Activity of anti-VEGF Antibodies

Cell-based Ligand-binding Assay

Anti-VEGF antibodies induced by vaccination with VEGF variants are tested for their ability to block the interaction between VEGF and VEGF receptor, by cell-based ligand-binding competition experiments. Briefly, VEGFR-2-expressing endothelial cells (such as the human umbilical vein endothelial cells (HUVEC) or the mouse H5V (H2^{d}) or bEND.3 (H2^{b}) endothelial cells) are grown in cell culture plates. Immune sera from VEGF variant—immunized animals and unlabeled recombinant VEGF are serially diluted and incubated with the cells. After washing steps, cells are incubated with [¹²⁵I]VEGF and specific radioactivity bound to the cells is determined.

Cell-based Proliferation Assay

Anti-VEGF antibodies are tested for their ability to inhibit the VEGF mitogenic activity on endothelial cells (such as the human umbilical vein endothelial cells (HUVEC) or the mouse H5V (H2^{d}) or bEND.3 (H2^{b}) endothelial cells). Endothelial cell proliferation can be monitored by ³H-thymidine incorporation for example.

### EXAMPLE 5

### Proof of VEGF Variant Efficacy in Animals

A variety of transformed cell lines express the VEGF mRNA and secrete VEGF. These cell lines can be used for demonstrating that anti-VEGF antibodies induced by the VEGF variant vaccine inhibit tumor growth in vivo.

Xenograft Models

Efficacy of the human VEGF variant vaccine is evaluated using Xenograft tumor models. Immunocompetent mice (or other immunocompetent hosts such as rats, rabbits, guinea pigs, monkeys) are vaccinated with human VEGF variant molecules formulated in an adequate adjuvant in order to induce an anti-VEGF antibody response. The VEGF antiserum generated in vaccinated animals, and containing anti-VEGF antibodies, is then transferred to immunodeficient mice (such as Nude or SCID mice) that have been transplanted with human tumor cells that express VEGF mRNA. Inhibition of tumor growth is measured by means of tumor size (mm²) and/or tumor volume (mm³) and/or tumor weight (mg). Different tumor cell lines can be used for these experiments such as the human A673 rhabdomyosarcoma, the SK-LMS1 leiomyosarcoma, and the SK-NEP-1 Wilms tumor cell lines.

Injection of the VEGF antiserum can be performed at different time-points relatively to the tumor challenge.

Syngeneic Models

Tumor models in mice allow direct vaccination of animals with mouse VEGF variant molecules and evaluation of the prophylactic as well as the therapeutic properties of the vaccine in an in vivo syngeneic tumor model.

Mice received one or more immunizations using different amounts of mouse VEGF variant molecules formulated in an adequate adjuvant. Tumor cells are injected either before or after mouse immunizations happen. Different tumor cell lines are available for such assays such as the mouse B16, B16F10.9 and B16G3.26 melanoma, the MC-38 and CT-26 colon carcinoma, and the D121 and D122-96 non-small cell lewis lung carcinoma cell lines. Inhibition of tumor growth is measured by means of tumor size (mm²) and/or tumor volume (mm³) and/or tumor weight (mg). Eventually, development of metastatic lesions (such as lung metastases) is also monitored.

### List of References

1. 2003. Phase III Trial of Avastin Plus Chemotherapy Markedly Extends Survival of Metastatic Colorectal Cancer Patients. Genentech Inc. Press release.
2. Takahashi, Y., Y. Kitadai, C. D. Bucana, K. R. Cleary, and L. M. Ellis. 1995. Expression of vascular endothelial growth factor and its receptor, KDR, correlates with vascularity, metastasis, and proliferation of human colon cancer. Cancer Res. 55:3964-3968.
3. Tokunaga, T., Y. Oshika, Y. Abe, Y. Ozeki, S. Sadahiro, H. Kijima, T. Tsuchida, H. Yamazaki, Y. Ueyama, N. Tamaoki, and M. Nakamura. 1998. Vascular endothelial growth factor (VEGF) mRNA isoform expression pattern is correlated with liver metastasis and poor prognosis in colon cancer. Br.J. Cancer 77:998-1002.
4. Kabbinavar, F., H. I. Hurwitz, L. Fehrenbacher, N. J. Meropol, W. F. Novotny, G. Lieberman, S. Griffing, and E. Bergsland. 2003. Phase II, randomized trial comparing bevacizumab plus fluorouracil (FU)/leucovorin (LV) with FU/LV alone in patients with metastatic colorectal cancer. J. Clin.Oncol. 21:60-65.
5. Ferrara, N. 2002. VEGF and the quest for tumour angiogenesis factors. Nat.Rev.Cancer 2:795-803.
6. Prewett, M., J. Huber, Y. Li, A. Santiago, W. O'Connor, K. King, J. Overholser, A. Hooper, B. Pytowski, L. Witte, P. Bohlen, and D. J. Hicklin. 1999. Antivascular endothelial growth factor receptor (fetal liver kinase 1) monoclonal antibody inhibits tumor angiogenesis and growth of several mouse and human tumors. Cancer Res. 59:5209-5218.
7. Zhu, Z., P. Rockwell, D. Lu, H. Kotanides, B. Pytowski, D. J. Hicklin, P. Bohlen, and L. Witte. 1998. Inhibition of vascular endothelial growth factor-induced receptor activation with anti-kinase insert domain-containing receptor single-chain antibodies from a phage display library. Cancer Res. 58:3209-3214.
8. Niethammer, A. G., R. Xiang, J. C. Becker, H. Wodrich, U. Pertl, G. Karsten, B. P. Eliceiri, and R. A. Reisfeld. 2002. A DNA vaccine against VEGF receptor 2 prevents effective angiogenesis and inhibits tumor growth. Nat.Med. 8:1369-1375.
9. Li, Y., M. N. Wang, H. Li, K. D. King, R. Bassi, H. Sun, A. Santiago, A. T. Hooper, P. Bohlen, and D. J. Hicklin. 2002. Active immunization against the vascular endothelial growth factor receptor flk1 inhibits tumor angiogenesis and metastasis. J.Exp.Med. 195:1575-1584.
10. Stopeck, A., M. Sheldon, M. Vahedian, G. Cropp, R. Gosalia, and A. Hannah. 2002. Results of a Phase I dose-escalating study of the antiangiogenic agent, SU5416, in patients with advanced malignancies. Clin.Cancer Res. 8:2798-2805.
11. Devore, R. F., L. Feherbacher, R. S. Herbst, C. J. Langer, K. Kelly, J. Gaudreault, E. Holmgren, W. F. Novotny, and F. Kabbinavar. 2000. A randomized phase II trial comparing Rhumab VEGF (recombinant humanized monoclonal antibody to vascular endothelial cell growth factor) plus carboplatin/paclitaxel (CP) to CP alone in patients with stage IIIB/IV NSCLC. Proceedings of the American Society for Clinical Oncology 19:485a. (Abstr.)
12. Yang, J . C., L. Haworth, S. M. Steinberg, S. A. Rosenberg, and W. Novotny. 2002. A randomized double-blind placebo-controlled trial of bevacizumab (anti-VEGF antibody) demonstrating a prolongation in time to progression in patients with metastatic renal cancer. Proceedings of the American Society for Clinical Oncology 21:1. (Abstr.)
13. Smolich, B. D., H. A. Yuen, K. A. West, F. J. Giles, M. Albitar, and J. M. Cherrington. 2001. The antiangiogenic protein kinase Inhibitors SU5416 and SU6668 inhibit the SCF receptor (c-kit) in a human myeloid leukemia cell line and in acute myeloid leukemia blasts. Blood 97:1413-1421.
14. Dias, S., K. Hattori, Z. Zhu, B. Heissig, M. Choy, W. Lane, Y. Wu, A. Chadburn, E. Hyjek, M. Gill, D. J. Hicklin, L. Witte, M. A. Moore, and S. Rafii. 2000. Autocrine stimulation of VEGFR-2 activates human leukemic cell growth and migration. J.Clin.Invest 106:511-521.
15. Cross,M. J., J. Dixelius, T. Matsumoto, and L. Claesson-Welsh. 2003. VEGF-receptor signal transduction. Trends Biochem.Sci. 28:488-494.
16. Muller, Y. A., H. W. Christinger, B. A. Keyt, and A. M. de Vos. 1997. The crystal structure of vascular endothelial growth factor (VEGF) refined to 1.93 A resolution: multiple copy flexibility and receptor binding. Structure. 5:1325-1338.
17. Muller, Y. A., B. Li, H. W. Christinger, J. A. Wells, B. C. Cunningham, and A. M. de Vos. 1997. Vascular endothelial growth factor: crystal structure and functional mapping of the kinase domain receptor binding site. Proc.Natl.Acad.Sci.U.S.A 94:7192-7197.
18. Wiesmann, C., G. Fuh, H. W. Christinger, C. Eigenbrot, J. A. Wells, and A. M. de Vos. 1997. Crystal structure at 1.7 A resolution of VEGF in complex with domain 2 of the Flt-1 receptor. Cell 91:695-704.
19. Wiesmann, C., H. W. Christinger, A. G. Cochran, B. C. Cunningham, W. J. Fairbrother, C. J. Keenan, G. Meng, and A. M. de Vos. 1998. Crystal structure of the complex between VEGF and a receptor-blocking peptide. Biochemistry 37:17765-17772.
20. Muller, Y. A., Y. Chen, H. W. Christinger, B. Li, B. C. Cunningham, H. B. Lowman, and A. M. de Vos. 1998. VEGF and the Fab fragment of a humanized neutralizing antibody: crystal structure of the complex at 2.4 A resolution and mutational analysis of the interface. Structure. 6:1153-1167.
21. Fairbrother,W. J., M. A. Champe, H. W. Christinger, B. A. Keyt, and M. A. Starovasnik. 1998. Solution structure of the heparin-binding domain of vascular endothelial growth factor. Structure. 6:637-648.
22. Christinger, H. W., Y. A. Muller, L. T. Berleau, B. A. Keyt, B. C. Cunningham, N. Ferrara, and A. M. de Vos. 1996. Crystallization of the receptor binding domain of vascular endothelial growth factor. Proteins 26:353-357.
23. Starovasnik, M. A., H. W. Christinger, C. Wiesmann, M. A. Champe, A. M. de Vos, and N. J. Skelton. 1999. Solution structure of the VEGF-binding domain of Fit-1: comparison of its free and bound states. J.Mol.Biol. 293:531-544.
24. Fiebich, B. L., B. Jager, C. Schollmann, K. Weindel, J. Wilting, G. Kochs, D. Marme, H. Hug, and H. A. Welch. 1993. Synthesis and assembly of functionally active human vascular endothelial growth factor homodimers in insect cells. Eur.J.Biochem. 211:19-26.
25. Meng, Y. G., I. Liang, W. L. Wong, and V. Chisholm. 2000. Green fluorescent protein as a second selectable marker for selection of high producing clones from transfected CHO cells. Gene 242:201-207.
26. Ferrara, N., J. Winer, T. Burton, A. Rowland, M. Siegel, H. S. Phillips, T. Terrell, G. A. Keller, and A. D. Levinson. 1993. Expression of vascular endothelial growth factor does not promote transformation but confers a growth advantage in vivo to Chinese hamster ovary cells. J.Clin.Invest 91:160-170.

1. A method for in vivo down-regulation of Vascular Endothelial Growth Factor (VEGF) activity in an animal, including a human being, the method comprising effecting presentation to the animal's immune system of an immunogenically effective amount of
at least one autologous VEGF protein or an autologous VEGF polypeptide or subsequence thereof which has been formulated so that immunization of the animal with the VEGF protein or VEGF polypeptide or subsequence thereof induces production of antibodies the animal's autologous VEGF protein, and/or
at least one VEGF analogue, which comprises a VEGF polypeptide wherein is introduced at least one modification in the VEGF amino acid sequence which has as a result that immunization of the animal with the analogue induces production of antibodies against the animal's autologous VEGF protein.

2. The method according to claim 1, wherein is presented a VEGF analogue with at least one modification of the VEGF amino acid sequence.

3. The method according to claim 2, wherein the modification has as a result that a substantial fraction of VEGF B-cell epitopes are preserved and that
at least one foreign T helper lymphocyte epitope (T_{H} epitope) is introduced, and/or
at least one first moiety is introduced which effects targeting of the modified molecule to an antigen presenting cell (APC) or a B-lymphocyte, and/or
at least one second moiety is introduced which stimulates the immune system, and/or
at least one third moiety is introduced which optimizes presentation of the modified VEGF polypeptide to the immune system.

4. The method according to claim 3, wherein the modification includes introduction as side groups, by covalent or non-covalent binding to suitable chemical groups in the VEGF polypeptide or a subsequence thereof, of the foreign T_{H} epitope and/or of the first and/or of the second and/or of the third moiety.

5. The method according to claim 3, wherein the modification includes amino acid substitution and/or deletion and/or insertion and/or addition.

6. The method according to claim 5, wherein the modification results in the provision of a fusion polypeptide.

7. The method according to claim 5, wherein introduction of the amino acid substitution and/or deletion and/or insertion and/or addition results in a substantial preservation of the 3D structure of the VEGF polypeptide.

8. The method according to claim 5, wherein introduction of the amino acid substitution and/or deletion and/or insertion and/or addition results in a substantial preservation of the overall quarternary structure of the autologous VEGF protein.

9. The method according to claim 2, wherein the modification includes duplication of at least one VEGF B-cell epitope and/or introduction of a hapten.

10. The method according to claim 3, wherein the foreign T-cell epitope is immunodominant in the animal.

11. The method according to claim 3, wherein the foreign T-cell epitope is promiscuous.

12. The method according to claim 11, wherein the at least one foreign T-cell epitope is selected from a natural promiscuous T-cell epitope and an artificial MHC-II binding peptide sequence.

13. The method according to claim 11, wherein the natural T-cell epitope is selected from a Tetanus toxoid epitope such as P2 or P30, a diphtheria toxoid epitope, an influenza virus hemagluttinin epitope, and a P. falciparum CS epitope, and wherein the artificial MHC-II binding peptide is a pan DR binding peptide.

14. The method according to claim 3, wherein the first moiety is a substantially specific binding partner for a B-lymphocyte specific surface antigen or for an APC specific surface antigen, such as a hapten or a carbohydrate for which there is a receptor on the B-lymphocyte or the APC.

15. The method according to claim 3, wherein the second moiety is selected from a cytokine, a hormone, and a heat-shock protein.

16. The method according to claim 15, wherein the cytokine is selected from, or is an effective part of, interferon γ (IFN-γ), Flt3L, interleukin 1 (IL-1), interleukin 2 (IL-2), interleukin 4 (IL-4), interleukin 6 (IL-6), interleukin 12 (IL-12), interleukin 13 (IL-13), interleukin 15 (IL-15), and granulocyte-macrophage colony stimulating factor (GM-CSF), and the heat-shock protein is selected from, or is an effective part of any of, HSP70, HSP90, HSC70, GRP94, and calreticulin (CRT).

17. The method according to claim 3, wherein the third moiety is of lipid nature, such as a palmitoyl group, a myristyl group, a farnesyl group, a geranyl-geranyl group, a GPI-anchor, and an N-acyl diglyceride group.

18. The method according to claim 17, wherein the VEGF polypeptide is a human VEGF-A polypeptide, preferably VEGF-A isoform 121 (SEQ ID NO: 5) or isoform 165 (SEQ ID NO: 4).

19. The method according to claim 18, wherein the human VEGF-A polypeptide has been modified within the N-terminal part proximal to the first beta-strand and/or within the C-terminus of the cystine knot domain and/or within the loop between beta-strands B3 and B4.

20. The method according to claim 19, wherein the human VEGF-A polypeptide has been modified by insertion into, deletionin, addition to, or substitution of any one of amino acids 1-15 in any one of SEQ ID NOs: 2-8.

21. The method according to claim 19, wherein the human VEGF-A polypeptide has been modified by insertion into, deletion in, addition to, or substitution of any amino acid C-terminal to residue 105 in any one of SEQ ID NOs: 2-8.

22. The method according to claim 19, wherein the human VEGF-A polypeptide has been modified by insertion, deletion or substitution in any one of SEQ ID NOs: 2-8, residues 59-66.

23. The method according to claim 1, wherein the analogue comprises a structure selected from
VEGFₘ-X-VEGF, VEGF-Xₘ-VEGF, VEGF-X-VEGFₙ, VEGFₘ-Xₘ-VEGF, VEGFₘ-X-VEGFₙ, VEGF-Xₘ-VEGFₙ, and VEGFₘ-Xₘ-VEGFₙ, wherein VEGF is a VEGF polypeptide or
subsequence thereof, X is an inert linker, VEGFₘ is a VEGF polypeptide or subsequence thereof that includes a modification constituting or contributing to the presence of the at least one foreign T helper epitope in the analogue, VEGFₙ is a VEGF polypeptide or subsequence thereof that includes a modification constituting or contributing to the presence of the at least one foreign T helper epitope in the analogue, and Xₘ is a peptide linker that includes or contributes to the presence of the at least one foreign T helper epitope in the analogue.

24. The method according to claim 23, wherein the analogue has the formula VEGF-Xₘ-VEGF.

25. The method according to claim 1, wherein presentation to the immune system is effected by having at least two copies of the VEGF polypeptide, the subsequence thereof or the modified VEGF polypeptide covalently of non-covalently linked to a carrier molecule capable of effecting presentation of multiple copies of antigenic determinants.

26. The method according to claim 1, wherein the VEGF polypeptide, the subsequence thereof, or the modified VEGF polypeptide has been formulated with an adjuvant which facilitates breaking of autotolerance to autoantigens.

27. The method according to claim 25, wherein the adjuvant is selected from the group consisting of an immune targeting adjuvant; an immune modulating adjuvant such as a toxin, a cytokine and a mycobacterial derivative; an oil formulation; a polymer; a micelle forming adjuvant; a saponin; an immunostimulating complex matrix (an ISCOM matrix); a particle; DDA; aluminium adjuvants; DNA adjuvants; γ-inulin; and an encapsulating adjuvant.

28. The method according to claim 1, wherein an effective amount of the VEGF polypeptide or the VEGF analogue is administered to the animal via a route selected from the parenteral route such as the intracutaneous, the subcutaneous, and the intramuscular routes; the peritoneal route; the oral route; the buccal route; the sublinqual route; the epidural route; the spinal route; the anal route; and the intracranial route.

29. The method according to claim 28, wherein the effective amount is between 0.5 μg and 2,000 μg of the VEGF polypeptide, the subsequence thereof or the analogue thereof.

30. The method according to claim 28, which includes at least one administration of the VEGF polypeptide or analogue per year, such as at least 2, at least 3, at least 4, at least 6, and at least 12 administrations per year.

31. The method according to claim 28, wherein the VEGF polypeptide or analogue is contained in a virtual lymph node (VLN) device.

32. The method according to claim 1, wherein presentation of modified VEGF to the immune system is effected by introducing nucleic acid(s) encoding the modified VEGF into the animal's cells and thereby obtaining in vivo expression by the cells of the nucleic acid(s) introduced.

33. The method according to claim 32, wherein the nucleic acid(s) introduced is/are selected from naked DNA, DNA formulated with charged or uncharged lipids, DNA formulated in liposomes, DNA included in a viral vector, DNA formulated with a transfection-facilitating protein or polypeptide, DNA formulated with a targeting protein or polypeptide, DNA formulated with Calcium precipitating agents, DNA coupled to an inert carrier molecule, DNA encapsulated in chitin or chitosan, and DNA formulated with an adjuvant such as the adjuvants.

34. The method according to claim 32, wherein the nucleic acids are administered intraarterially, intraveneously, or by the routes.

35. The method according to claim 33, wherein the nucleic acid(s) is/are contained in a VLN device.

36. The method according to claim 33, which includes at least one administration of the nucleic acids per year, such as at least 2, at least 3, at least 4, at least 6, and at least 12 administrations per year

37. A method for treating and/or preventing and/or ameliorating diseases selected from the group consisting of malignant neoplasm, benign neoplasm, inflammatory diseases, and diabetes and diabetes related conditions, the method comprising down-regulation of VEGF according to claim 1.

38. A VEGF analogue which is derived from an animal VEGF polypeptide wherein is introduced a modification which has as a result that immunization of the animal with the analogue induces production of antibodies against the VEGF polypeptide, and wherein the modification is as defined in claim 1.

39. An immunogenic composition comprising an immunogenically effective amount of a VEGF polypeptide autologous in an animal, said VEGF polypeptide being formulated together with an immunologically acceptable adjuvant so as to break the animal's autotolerance towards the VEGF polypeptide, the composition further comprising a pharmaceutically and immunologically acceptable carrier and/or vehicle.

40. An immunogenic composition comprising an immunogenically effective amount of a VEGF analogue according to claim 38, the composition further comprising a pharmaceutically and immunologically acceptable carrier and/or vehicle and optionally an adjuvant.

41. An immunogenic composition according to claim 39, wherein the adjuvant is selected from the group consisting of the adjuvants.

42. A nucleic acid fragment which encodes a VEGF analogue according to claim 38.

43. A vector carrying the nucleic acid fragment according to claim 42.

44. The vector according to claim 43 which is capable of autonomous replication.

45. The vector according to claim 43 which is selected from the group consisting of a plasmid, a phage, a cosmid, a mini-chromosome, and a virus.

46. The vector according to claim 43, comprising, in the 5′→3′ direction and in operable linkage, a promoter for driving expression of the nucleic acid fragment, optionally a nucleic acid sequence encoding a leader peptide enabling secretion of or integration into the membrane of the polypeptide fragment, the nucleic acid fragment, and optionally a terminator.

47. The vector according to claim 43 which, when introduced into a host cell, is integrated in the host cell genome.

48. The vector according to claim 43 which, when introduced into a host cell, is not capable of being integrated in the host cell genome.

49. The vector according to claim 43, wherein the promoter drives expression in a eukaryotic cell and/or in a prokaryotic cell.

50. A transformed cell carrying the vector of claim 43.

51. The transformed cell according to claim 50 which is capable of replicating the nucleic acid fragment.

52. The transformed cell according to claim 51, which is a microorganism selected from a bacterium, a yeast, a protozoan, or a cell derived from a multicellular organism selected from a fungus, an insect cell such as an S2 or an SF cell, a plant cell, and a mammalian cell.

53. The transformed cell according to claim 52 which is a bacterium of the genus Escherichia, Bacillus, Salmonella, or Mycobacterium.

54. The transformed cell according to claim 53, which is selected from the group consisting of an E. coli cell, and a non-pathogenic Mycobacterium cell such as M. bovis BCG.

55. The transformed cell according to claim 50, which expresses the nucleic acid fragment.

56. The transformed cell according to claim 55, which secretes or carries on its surface, the VEGF analogue.

57. The method according to claim 1, wherein presentation to the immune system is effected by administering a non-pathogenic microorganism or virus which is carrying a nucleic acid fragment which encodes and expresses the VEGF polypeptide or analogue.

58. The method according to claim 57, wherein the virus is a non-virulent pox virus such as a vaccinia virus.

59. The method according to claim 58, wherein the microorganism is a bacterium, such as a bacterium.

60. The method according to claim 57, wherein the non-pathogenic microorganism or virus is administered one single time to the animal.

61. A composition for inducing production of antibodies against VEGF, the composition comprising
a nucleic acid fragment according to claim 42 or a vector, and
a pharmaceutically and immunologically acceptable carrier and/or vehicle and/or adjuvant.

62. The composition according to claim 61, wherein the nucleic acid fragment is formulated.

63. A stable cell line which carries the vector according to claim 43 and which expresses the nucleic acid fragment, and which optionally secretes or carries the VEGF analogue on its surface.

64. A method for the preparation of the cell according to claim 50, the method comprising transforming a host cell with the nucleic acid fragment according to claim 42 or with the vector according to any one of claims 43-49.
